(19) United States
(12) Patent Application Publication
(10) Pub. No.: US 2003/0059436 A1
(43) Pub. Date: Mar. 27, 2003

(54) Band 3 antigenic peptides, malaria polypeptides and uses thereof

(75) Inventors: Athar H. Chishti, Sudbury, MA (US); S. Steven Oh, Hopkinton, MA (US); David Liu, Newton, MA (US); Vikas Goel, Brighton, MA (US); Xuerong Li, Boston, MA (US)

Correspondence Address: Elizabeth R. Plumer, c/o Wolf, Greenfield & Sacks, P.C., Federal Reserve Plaza, 600 Atlantic Avenue, Boston, MA, 02210-2211, US

(73) Assignee: St. Elizabeth's Medical Center, Inc., Boston, MA

(21) Appl. No.: 10/087,464

(22) Filed: Mar. 1, 2002

Related U.S. Application Data
(60) Provisional application No. 60/272,930, filed on Mar. 2, 2001.

(51) Int. Cl. A61K 39/00; C07K 14/435; C12N 5/06

(52) U.S. Cl. 424/185.1; 530/326; 514/14; 435/325; 435/320.1

(57) ABSTRACT

The invention provides peptides derived from erythroid Band 3 protein, which selectively bind to merozoite surface protein-1 (MSP-1), and/or one or more of the malaria polypeptides: BBP-1, BBP-2, BBP-3, BBP-4, BBP-5, BBP-6, RhopH3, and ABRA and prevent infection by the parasite of a Band 3-expressing cell, such as an erythrocyte. The invention also provides the isolated polypeptides BBP-1, BBP-2, BBP-3, BBP-4, BBP-5, BBP-6, RhopH3, and/or ABRA as well as peptides derived from MSP-1, which selectively bind to erythroid Band 3 protein and prevent parasite invasion into a Band 3-expressing cell, and prevent Plasmodium infection. Methods of using the malaria and MSP1 polypeptides of the invention for malaria prevention and/or treatment (e.g. in vaccines) are also provided. Antibodies that bind to the Band 3 polypeptides and anti-idiotypic antibodies thereto also are provided. Methods for selecting agents which inhibit Band 3-mediated parasite entry into target cells and methods of treatment which involve the polypeptides, antibodies, and anti-idiotypic antibodies also are provided.

### RELATED APPLICATIONS

This application claims priority under 35 U.S.C. §119 to U.S. No. 60/272,930, filed Mar. 2, 2001, the entire contents of which is hereby incorporated by reference.

### GOVERNMENT SUPPORT

This invention was made in part with government support under grant number HL60961 and HL60755 from the National Institutes of Health (NIH). The government may have certain rights in this invention.

### FIELD OF THE INVENTION

This invention relates to polypeptides derived from erythroid Band 3 protein and nucleic acid molecules encoding same. The polypeptides selectively bind to merozoite surface protein-1 (MSP-1) and/or to one or more of the polypeptides: BBP-1, BBP-2, BBP-3, BBP-4, BBP-5, BBP-6, RhopH3, and ABRA. The invention also relates in part to nucleic acids that encode the polypeptides BBP-1, BBP-2, BBP-3, BBP-4, BBP-5, BBP-6, RhopH3, and ABRA. The invention also relates to polypeptides derived from MSP-1 which selectively bind to Band 3 protein and nucleic acid molecules encoding same. The nucleic acid molecules and encoded polypeptides are useful in, inter alia, research, diagnostic and therapeutic contexts, particularly for the development of antibodies and anti-idiotypic antibodies for treating malaria infection.

### BACKGROUND OF THE INVENTION

The World Health Organization estimates that 300-500 million people are infected by malaria annually and over 2 million people, mostly women and children under the age of five die of the malaria disease each year. The disease has been classified as an “emerging infection” by many national and international health authorities in recent years, due to its dramatic comeback in regions where the disease is once eliminated or suppressed. Conventional method of control for malaria disease mainly relies on the use of antimalarial drugs. Due to a rapid rise and spread of drug resistance to most affordable and widely used drugs in recent years, however, there is unfortunately limited means of treatment for the disease. At present, a malaria vaccine is not available.

In view of the foregoing, a need exists to develop new and improved methods and compositions for treating malaria infection. Preferably such methods and compositions are based upon inhibiting the particular interactions between the malaria parasite and a cognate molecule present in the host, thereby minimizing harmful side effects that may be due to non-specific therapeutic approaches.

### SUMMARY OF THE INVENTION

The invention is based, in part, on our discovery that the erythrocyte Band 3 protein is an important receptor for malaria parasite invasion into host erythrocytes. Important regions of the Band 3 protein that form the receptor in human erythrocytes are defined as amino acid residues 720-761 in the ectoplasmic domain 5 and residues 807-826 in the ectoplasmic domain 6. These two ectoplasmic domains of the erythroid Band 3 protein appear to be an important part of the erythrocyte receptor or receptor complex required for the P. falciparum invasion of the erythrocytes.

It now has been discovered that particular sequences within the erythroid Band 3 protein (also known as Anion Exchanger 1 or AE1) selectively interact with merozoite surface protein-1 (MSP-1) protein, resulting in entry of the malaria parasite into the erythrocyte host cell. In addition, other polypeptides that selectively interact with the Band-3 derived sequences have been identified. These polypeptides include: BBP-1, BBP-2, BBP-3, BBP-4, BBP-5, BBP-6, RhopH3, and ABRA. Accordingly, these particular Band 3-derived sequences are useful for further defining the nature of the interaction(s) between the parasite and the erythrocyte which result in infection, as well as for developing diagnostic and therapeutic agents which are useful for detecting and treating malaria infection. The knowledge of the particular sequences of the Band 3 protein which are important to malaria infection also permits the development of novel anti-idiotypic agents for treating malaria infection. These aspects of the invention are summarized below.

The invention also is based, in part, on the discovery that the polypeptides BBP-1, BBP-2, BBP-3, BBP-4, BBP-5, BBP-6, RhopH3, ABRA, and particular sequences within MSP-1 selectively interact with Band 3 protein. Accordingly, the polypeptides BBP-1, BBP-2, BBP-3, BBP-4, BBP-5, BBP-6, RhopH3, ABRA and these particular MSP-1 sequences are useful as targets for developing diagnostic and therapeutic agents for detecting and treating malaria infection. These aspects of the invention are summarized below.

In view of the foregoing discoveries, the invention embraces methods for inhibiting the selective interaction between the Band 3 protein and one or more of the polypeptides BBP-1, BBP-2, BBP-3, BBP-4, BBP-5, BBP-6, RhopH3, ABRA, and MSP-1, as well as related compositions. Such methods are useful for identifying compounds for therapeutic use (e.g., screening assays), as well as for diagnosing and/or treating a malaria infection.

In addition, the invention also relates in part to nucleic acids that encode the polypeptides BBP-1, BBP-2, BBP-3, BBP-4, BBP-5, BBP-6, RhopH3, ABRA and MSP-1.

According to one aspect of the invention, isolated Band 3 polypeptides are provided. The isolated Band 3 polypeptides include amino acid sequences selected from the group consisting of SEQ ID NO. 1, 2, 3, and 4 as shown herein: SEQ ID NO: 1: GMPWLSATTVRSVTHANALT (also referred to herein as sequence B3_{5A}); SEQ ID NO:2: SVTHANALTVMGKASTPGAA (also referred to herein as sequence B3_{5B}); SEQ ID NO:3: GKASTPGAAAQIQEVKEQRI (also referred to herein as sequence B3_{5C}); SEQ ID NO:4:DRILLLFKPPKYHPDVPYVK (also referred to herein as sequence B3_{6A}); and unique fragments thereof, wherein the unique fragments (1) bind to an MSP-1 polypeptide and (2) exclude the sequences set forth in Table 4:Band 3 Blast Homology Sequences. According to another aspect of the invention, isolated nucleic acid molecules that encode the foregoing polypeptides are provided. According to yet another aspect of the invention, expression vectors that include the foregoing nucleic acid molecules operably linked to a promoter are provided. In another aspect of the invention host cells transfected or transformed with the expression vector are provided.

According to another aspect of the invention, immunogenic compositions are provided. The compositions include one or more of the foregoing isolated Band 3 polypeptides; and a pharmaceutically acceptable carrier; wherein the polypeptides are present in an effective amount to induce an immune system response. In some embodiments, the compositions also include an adjuvant. According to another aspect of the invention, methods of making a medicament are provided. The methods include placing one or more of the foregoing isolated Band 3 polypeptides in a pharmaceutically acceptable carrier.

According to yet another aspect of the invention, methods for identifying a candidate mimetic of the foregoing isolated Band 3 polypeptides are provided. The methods include providing an MSP-1 polypeptide which binds a foregoing Band 3 polypeptide, contacting the MSP-1 polypeptide with a test molecule, and determining the binding of the test molecule to the MSP-1 polypeptide, wherein a test molecule which binds to the MSP-1 polypeptide and inhibits binding of the MSP-1 polypeptide to the foregoing isolated Band 3 polypeptide is a candidate mimetic of the foregoing isolated Band 3 polypeptide.

According to another aspect of the invention, protein microarrays are provided. The microarrays include at least one isolated Band 3 polypeptide selected from the group consisting of SEQ ID NOS. 1, 2, 3, and 4.

According to yet another aspect of the invention, anti-Band 3 antibodies or fragments thereof are provided. The anti-Band 3 antibodies or fragments thereof selectively bind to a foregoing isolated Band 3 polypeptide, wherein the antibody inhibits infection of cells by P. falciparum merozoite malaria parasite. Preferably, the antibody is a monoclonal antibody, and, more preferably, a humanized monoclonal antibody.

According to another aspect of the invention, anti-idiotype antibodies which selectively bind to an idiotype of the foregoing Band 3 antibodies are provided. As used herein, an idiotype refers to a specific binding site of an antibody that binds to the peptide antigen. In accordance with the present invention, the anti-idiotype antibody blocks penetration of malaria parasite into human red blood cells, presumably by virtue of blocking the malarial parasite ligand that binds to the erythroid Band 3 protein receptor. According to a related aspect of the invention, methods for making an anti-idiotypic antibody are provided. The methods include immunizing an animal with a foregoing Band 3 antibody under conditions to elicit an immune system response to an idiotype of said foregoing antibody.

According to some aspects of the invention, methods for treating a malaria infection, are provided. The methods include administering to a subject in need of such treatment, an effective amount of a foregoing anti-Band 3 antibody to treat the malaria infection. This method of treatment is referred to herein as “passive immunity”.

According to another aspect of the invention, methods for inducing an immune system response to treat a malaria infection are provided. The methods include administering to a subject in need of such treatment, an effective amount of a foregoing anti-Band 3 antibody under conditions to induce an anti-idiotypic immune response to the anti-Band 3 antibody idiotype. This method of treatment is referred to herein as “active immunity”.

The invention also is based, in part, on the discovery of the particular portion of MSP-1 (alternatively referred to herein as “MSP1”) that selectively binds to Band 3 protein. Thus the invention embraces various compositions containing such MSP-1 peptides for use, e.g., in screening assays to detect the specific interaction between MSP-1 and Band 3 protein, as well as for use in diagnostic and therapeutic applications for detecting and treating, respectively, malaria infection. In general, such compositions contain components (or are contained in kits which contain additional components) which are selected to detect the specific interaction between MSP-1 and Band 3 protein (particularly the Band 3 polypeptides disclosed herein).

According to another aspect of the invention, methods for identifying a candidate mimetic of a MSP-1 polypeptide are provided. The methods include providing an isolated Band 3 polypeptide which binds a MSP-1 polypeptide, contacting the Band 3 polypeptide with a test molecule, and determining the binding of the test molecule to the Band 3 polypeptide, wherein a test molecule which binds to the isolated Band 3 polypeptide and inhibits binding of the Band 3 polypeptide to the MSP-1 polypeptide is a candidate mimetic of the MSP-1 polypeptide. In some embodiments, the MSP-1 polypeptide has a sequence selected from the group consisting of SEQ ID NO: 11, SEQ ID NO: 12, SEQ ID NO: 13, SEQ ID NO:33, SEQ ID NO. 34, and SEQ ID NO:35. In certain embodiments, the test molecule is an antibody.

According to another aspect of the invention, isolated polypeptides are provided. The polypeptides include an amino acid sequence selected from the group consisting of SEQ ID NOs:11, 12, 13, 33, 34, and 35, or fragments thereof. According to another aspect of the invention, pharmaceutical compositions are provided. The compositions include one or more of the foregoing isolated MSP-1 polypeptides and a pharmaceutically acceptable carrier; wherein the polypeptides are present in an effective amount to induce an immune system response. In some embodiments, the pharmaceutical composition also includes an adjuvant. According to another aspect of the invention, methods of making a medicament are provided. The methods include placing one or more of the foregoing isolated MSP-1 polypeptides in a pharmaceutically acceptable carrier.

According to yet another aspect of the invention, methods of preventing or treating a malaria infection are provided. The methods include administering a foregoing MSP-1 pharmaceutical composition to a subject in need of such treatment in an amount effective to prevent or treat the malaria infection. According to some aspects of the invention, malaria polypeptide binding polypeptides are provided. The malaria polypeptide binding polypeptides selectively binds to a foregoing isolated MSP-1 polypeptide. Preferably, the binding polypeptide is an antibody or antigen-binding fragment of an antibody. Preferably, the antibody is a monoclonal antibody, and more preferably, a humanized monoclonal antibody. According to another aspect of the invention, pharmaceutical compositions that include the foregoing malaria polypeptide binding polypeptide in a pharmaceutically acceptable carrier are provided.

According to another aspect of the invention, methods of preventing or treating a malaria infection are provided. The methods include administering the foregoing MSP-1 polypeptide binding polypeptide pharmaceutical composition to a subject in need of such treatment in an amount effective to prevent or treat the malaria infection.

According to another aspect of the invention, isolated nucleic acids are provided. The isolated nucleic acids include a nucleotide sequence selected from the group consisting of SEQ ID NOs:54-59, or fragments thereof.

According to another aspect of the invention, an isolated Band 3 polypeptide is provided. The Band 3 polypeptide includes an amino acid sequence selected from the group consisting of SEQ ID NO. 1, 2, 3, and 4 as shown herein: SEQ ID NO: 1: GMPWLSATTVRSVTHANALT (also referred to herein as sequence B3_{5A}); SEQ ID NO:2: SVTHANALTVMGKASTPGAA (also referred to herein as sequence B3_{5B}); SEQ ID NO:3: GKASTPGAAAQIQEVKEQRI (also referred to herein as sequence B3_{5C}); SEQ ID NO:4: DRILLLFKPPKYHPDVPYVK (also referred to herein as sequence B3_{6A}), and unique fragments thereof, wherein the unique fragments (1) bind to an isolated polypeptide comprising an amino acid sequence selected from the group consisting of SEQ ID NOs:46-53, or fragment thereof, and (2) exclude the sequences set forth in Table 4:Band 3 Blast Homology Sequences.

According to another aspect of the invention, isolated nucleic acid molecules that encode the foregoing isolated Band 3 polypeptides are provided. According to another aspect of the invention, expression vectors are provided. The expression vectors include the foregoing isolated Band 3 nucleic acids operably linked to a promoter. According to another aspect of the invention, host cells transfected or transformed with the foregoing expression vectors are provided.

According to another aspect of the invention, immunogenic compositions are provided. The compositions include one or more of the foregoing isolated Band 3 polypeptides and a pharmaceutically acceptable carrier; wherein the Band 3 polypeptides are present in an effective amount to induce an immune system response. In some embodiments, the compositions also include an adjuvant. According to another aspect of the invention, methods of making a medicament are provided. The methods include placing one or more of the foregoing isolated Band 3 polypeptides in a pharmaceutically acceptable carrier.

According to another aspect of the invention, methods for identifying a candidate mimetic of a foregoing isolated Band 3 polypeptide are provided. The methods include providing a malaria polypeptide comprising an amino acid sequence selected from the group consisting of SEQ ID NOs:46-53, or fragment thereof that binds the foregoing isolated Band 3 polypeptide or fragment thereof, contacting the malaria polypeptide or fragment thereof, with a test molecule, and determining the binding of the test molecule to the malaria polypeptide or fragment thereof, wherein a test molecule which binds to the polypeptide or fragment thereof and inhibits binding of the foregoing isolated Band 3 polypeptide to the malaria polypeptide, is a candidate mimetic of the foregoing isolated Band 3 polypeptide.

The invention also is based, in part, on the discovery of BBP-1, BBP-2, BBP-3, BBP-4, BBP-5, BBP-6, RhopH3, and ABRA polypeptides that selectively binds to Band 3 protein. Thus the invention embraces various compositions containing such BBP-1, BBP-2, BBP-3, BBP-4, BBP-5, BBP-6, RhopH3, and ABRA polypeptides for use, e.g., in screening assays to detect the specific interaction between the BBP-1, BBP-2, BBP-3, BBP-4, BBP-5, BBP-6, RhopH3, and ABRA polypeptides and Band 3 protein, as well as for use in diagnostic and therapeutic applications for detecting and treating, respectively, malaria infection. In general, such compositions contain components (or are contained in kits which contain additional components) which are selected to detect the specific interaction between BBP-1, BBP-2, BBP-3, BBP-4, BBP-5, BBP-6, RhopH3, or ABRA and Band 3 protein (particularly the Band 3 polypeptides disclosed herein).

According to another aspect of the invention, methods for identifying a candidate mimetic of an isolated malaria polypeptide are provided. The methods include providing a Band 3 molecule which binds a malaria polypeptide comprising an amino acid sequence selected from the group consisting of SEQ ID NOs:46-53, contacting the Band 3 molecule with a test molecule, and determining the binding of the test molecule to the Band 3 molecule, wherein a test molecule which binds to the Band 3 molecule and inhibits binding of the malaria polypeptide with the Band 3 polypeptide is a candidate mimetic of the malaria polypeptide. In some embodiments, the test molecule is an antibody.

According to another aspect of the invention, isolated polypeptide molecules that include amino acid sequences selected from the group consisting SEQ ID NOs:46-52 are provided. According to another aspect of the invention, pharmaceutical compositions are provided. The pharmaceutical compositions include one or more of the foregoing isolated polypeptides and a pharmaceutically acceptable carrier; wherein the polypeptides are present in an effective amount to induce an immune system response. In some embodiments, the pharmaceutical composition also includes an adjuvant. According to another aspect of the invention, methods of making a medicament are provided. The methods include placing one or more of the foregoing isolated polypeptides in a pharmaceutically acceptable carrier. According to another aspect of the invention, methods of preventing or treating a malaria infection are provided. The methods include administering the foregoing pharmaceutical composition to a subject in need of such treatment in an amount effective to prevent or treat the malaria infection.

According to another aspect of the invention, malaria polypeptide binding polypeptides are provided. The malaria polypeptide-binding polypeptides selectively bind to the foregoing isolated malaria polypeptides, e.g., the binding polypeptide is an antibody or antigen-binding fragment of an antibody. Preferably, the antibody is a monoclonal antibody, and more preferably, a humanized monoclonal antibody. According to another aspect of the invention, pharmaceutical compositions that include the foregoing malaria polypeptide binding polypeptide in a pharmaceutically acceptable carrier are provided.

According to another aspect of the invention, methods of preventing or treating a malaria infection are provided. The methods include administering the foregoing pharmaceutical composition to a subject in need of such treatment in an amount effective to prevent or treat the malaria infection.

According to another aspect of the invention, isolated nucleic acid molecules are provided. The nucleic acid molecules are selected from the group consisting of:

(a) nucleic acid molecules which hybridize under stringent conditions to a nucleic acid molecule comprising a nucleotide sequence selected from the group consisting of SEQ ID NO:38-44 and which codes for a Plasmodium polypeptide,

(b) deletions, additions and substitutions of the nucleic acid molecules of (a), which code for a Plasmodium polypeptide,

(c) nucleic acid molecules that differ from the nucleic acid molecules of (a) or (b) in codon sequence due to the degeneracy of the genetic code, and

(d) complements of (a), (b) or (c).

In some embodiments, the isolated nucleic acid molecule comprises a nucleotide sequence selected from the group consisting of SEQ ID NO:38-44.

According to another aspect of the invention, isolated nucleic acid molecules are provided. The nucleic acid molecules are selected from the group consisting of:

(a) a unique fragment of the nucleotide sequence selected from the group consisting of:

nucleotides 1-1287 of SEQ ID NO:38 between 12 and 1286 nucleotides in length,

nucleotides 1-3576 of SEQ ID NO:39 between 12 and 3557 nucleotides in length,

nucleotides 1-903 of SEQ ID NO:40 between 12 and 902 nucleotides in length,

nucleotides 1-1203 of SEQ ID NO:41 between 12 and 1202 nucleotides in length,

nucleotides 1-3996 of SEQ ID NO:42 between 12 and 3995 nucleotides in length, and

nucleotides 1-876 of SEQ ID NO:43 between 12 and 875 nucleotides in length, and

nucleotides 1-2712 of SEQ ID NO:44 between 12 and 2711 nucleotides in length, and

(b) complements of (a),

wherein the unique fragments exclude nucleic acids having nucleotide sequences that are contained within SEQ ID NO:38-44, and that are known as of the filing date of this application.

According to another aspect of the invention, expression vectors that include the foregoing isolated nucleic acid molecules operably linked to a promoter are provided.

According to another aspect of the invention, isolated polypeptide molecules that include a unique fragment of amino acid sequence SEQ ID NO:53 that binds to a Band 3 polypeptide are provided.

These and other objects of the invention will be described in further detail in connection with the detailed description of the invention.

### BRIEF DESCRIPTION OF THE DRAWINGS

The figures are illustrative only and are not required for enablement of the inventions disclosed herein.

FIG. 1 is a bar graph (FIG. 1A) depicting P. falciparum infection of mouse RBCs in vitro and a digitized image of a photomicrograph (FIG. 1B) showing in Giemsa-stained thin smears at the end of the 24 h culture. The arrowhead indicates a newly infected RBC (ring stage parasite). The parasite culture was kept for 22-24 h at 37° C. as described (Klotz, F. W., et al., J Exp Med 165:1713-1718, 1987). Rings were counted from 5,000 RBCs in Giemsa-stained thin smears. Data analyzed as the mean of triplicate experiments with standard error.

FIG. 2 depicts the inhibition of P. falciparum invasion into RBCs by human Band 3 peptides. FIG. 2A shows a domain map illustration in (ii)-(iv), an inclusive boundary for each putative ectodomain was chosen as shown by vertical dotted lines. Ectodomains are shown as boxes in the three models. (i) Overlapping 12 to 20-residue peptides shown in solid bars were prepared according to the putative ectodomain boundaries. (ii) Casey model (Fujinaga, J., et al., J Biol Chem 274:6626-6633, 1999). (iii) Reithmeier model (Popov, M., et al., J Biol Chem 272:18325-18332, 1997). (iv) Sherman model (Crandall, I., et al., Parasitology 108:257-267, 1994). FIG. 2B shows a bar graph depicting an invasion inhibition assay by visual counting method, in which the number of ring stage parasites in 1,300-1,600 RBCs in Giemsa-stained thin smears was scored and plotted for each sample. Artemisinin (25 μM) and no peptide samples were positive and negative controls, respectively. FIG. 2C is a bar graph depicting of invasion inhibition assay by ³H-hypoxanthine incorporation method. Effects of Band 3 peptides relative to the control sample (no peptide) are shown. Artemisinin (50 μM) and unrelated peptide derived from dematin were used as positive and negative control, respectively. FIG. 2D is a bar graph that depicts results of a growth inhibition assay by ³H-hypoxanthine incorporation method. Effects of Band 3 peptides relative to the control sample (no peptide) are shown. In all inhibition assays, mean parasitemia from three experiments was calculated with standard error. DMSO background was corrected when necessary. Student's t test was used to compare with the control (no peptide). FIG. 2E is a table providing a summary of net charge and pI for Band 3 peptides.

FIG. 3 shows results of binding of human Band 3 and native P. falciparum merozoite proteins. FIG. 3A is a digitized photomicrographic image of a Giemsa-stained smear depicting purified merozoites with malaria pigments but no contaminating RBC components. FIG. 3B is a digitized image of a merozoite protein separation. Total merozoite proteins from purified merozoites and human RBC ghost proteins were separated by SDS-PAGE (M, merozoite; G, RBC ghost). Residual human serum albumin (HSA; apparent mass 67 kDa) from the culture medium is marked with an asterisk in the Coomassie gel. FIG. 3C is a digitized image of a blot overlay assay. The 5C+6A mixture specifically bound to merozoite proteins (arrowheads) but not RBC proteins on the blot. Peptides 3A, 4A, and 2 as negative control showed non-specific bindings to HSA (asterisk) in M and α/β spectrin (240/220 kDa) in G. FIG. 3D is a digitized image of a gel depicting recombinant 5ABC and 5BC expressed in E. coli. Coomassie gel (lanes 1-3) and anti-GST Western blot (lanes 4-6) showed affinity purified GST-5BC (lanes 1, 4) and GST-5ABC (lanes 2, 5). GST control sample (lanes 3, 6). FIG. 3E is a digitized image demonstrating native MSP1 binding to 5ABC. Autoradiography showed radiolabeled MSP1 (full length) and MSP1₄₂ bound to GST-5ABC (lane 1) and mAb 5.2 (lane 3), but not to GST (lane 2). Results reproduced three times.

FIG. 4 shows binding of recombinant human Band 3 and P. falciparum MSP1. FIG. 4A is a digitized image of a Coomassie gel showing recombinant MSP1 and Band 3 (lanes 1-3) and a digitized image of an anti-GST Western blot (lanes 4-6) showing affinity purified GST-MSP1₄₂ (lanes 1, 4), truncated GST-MSP1₃₈ (lanes 2, 5), and GST as control (lanes 3, 6). Truncations of GST-MSP1₃₈ appear to be at the C-terminus as anti-GST antibody reacted with all three major Bands. GST-MSP1₁₉ was co-purified with GST (lane 7, Coomassie; lane 8, anti-GST blot; lane 9, anti-mAb 5.2 blot). FIG. 4A also depicts a digitized image of an autorad showing ³²P-labeled MSP1₁₉ (lane 10), 5BC (lane 11), and 5ABC (lane 12). FIGS. 4B, 4C, and 4D show line graphs and a bar graph depicting results of a solution-binding assay. Binding assays were performed as described in Example 5 and in Oh, S. S., et al., Mol Biochem Parasitol 108:237-247, 2000. ³²P-labeled 5ABC (10, 20, 40, 80 μM) and 5BC (21, 42, 84, 168 μM) respectively bound to GST-MSP1₄₂ (FIG. 4B) and GST-MSP1₃₈ (FIG. 4C) on beads in concentration-dependent manner. ³²P-labeled MSP1₁₉ bound specifically to the 5ABC domain (FIG. 4D) as statistically analyzed by Student's t test. FIG. 4E is a graphical summary of MSP1 -Band 3 interactions. Dotted line denotes the C-terminal truncation of MSP1₃₈.

FIG. 5. depicts results indicting P. falciparum MSP1 binds to intact RBCs in suspension. FIG. 5A is a digitized image of a 10% Coomassie gel of enzyme-treated RBCs (as described in Example 5). The gel (lanes 1-6) shows ghost membrane proteins prepared from untreated human (lane 1) and mouse (lane 4) RBCs, Nm-treated human (lane 2) and mouse (lane 5) RBCs, and ChT-treated (40 min) human (lane 3) and mouse (lane 6) RBCs. Arrowheads and arrows respectively indicate full-length and ChT-digested Band 3. The middle panel is a digitized image of a gel demonstrating PAS staining of the gel, which allowed analysis of sialic acid content in RBC ghosts prepared from untreated (lane 7), ChT-treated (lane 8), and Nm-treated (lane 9) human RBC samples. The right panel depicts a digitized image of a Western blot of ghost proteins prepared from untreated (lanes 10, 13), ChT-treated (lanes 11, 14), and Nm-treated (lane 12) human RBCs using anti-Band 3 and anti-GPA antibody are shown. FIG. 5B is a digitized image of an anti-GST Western blot showing Nm-treated (lane 1) and untreated (lane 3) human RBCs binding GST-MSP1₃₈. GST was used as control (lanes 2, 4). FIG. 5C is a digitized image depicting ³²P-labeled MSP1₁₉ binding to various RBC types. Assays were repeated 3-6 times. Means (±standard error) were plotted relative to the control (untreated wild-type RBCs) and compared using Student's t test.

FIG. 6. is a digitized image of an invasion pathway model. In both sialic acid-independent and dependent pathways, Band 3 may function as an important RBC receptor for P. falciparum invasion. In the former pathway, Band 3 might be an independent receptor (open arrows) or complemented by GPA to take part in the sialic acid-dependent pathway (dotted arrow). In the latter pathway, GPA appears to be a non-essential receptor perhaps requiring the coupling of the essential Band 3 receptor (solid black arrows).

### DETAILED DESCRIPTION OF THE INVENTION

For over two decades, the malaria parasite protein MSP-1 has been reported to play an important role during the parasite invasion of red blood cells based on the findings that (a) it is a major protein found on the surface of the merozoite, the invasive form of the malaria parasite, (b) full-length MSP-1, a segment of MSP1(38), as well as a number of 20-mer peptides derived from MSP-1 bound to erythrocytes, (c) MSP-1 induced antibodies provided protection against malaria infection in animal models, and (d) deleting a portion of the MSP-1 gene caused destruction of the parasite in vitro. However, the exact function of MSP-1 has remained unknown. We have identified for the first time a definitive function of MSP-1 in the malaria parasite invasion of red blood cells. This newly identified function revealed that specific proteolytic fragments of MSP-1 (i.e., MSP1(38), MSP1(42), MSP1(19)) are the parasite ligands specifically binding to the erythrocyte receptor Band 3 during invasion and that the binding interaction between the parasite MSP-1 fragments and the host Band 3 receptor is important for the invasion process to proceed successfully. We have identified that MSP1 (19)—the 19 kDa C-terminal fragment of MSP-1 formed by secondary proteolytic processing of MSP1(42)—is also a parasite ligand binding to the Band 3 receptor peptides, e.g., SEQ ID NOs 1, 2, 3, and 4.

The functional form of MSP-1 during malaria parasite invasion into erythrocytes is not full-length MSP-1 (approximately 195-205 kDa depending upon the Plasmodium falciparum malaria strain) but its naturally processed proteolytic fragments generally known as MSP1(83), MSP1(30), MSP1(38), MSP1(42), and MSP1(33), and MSP1(19). The first four proteolytic fragments are formed upon primary processing of full-length MSP1. The last two are formed by secondary processing of MSP1 (42). These primary and secondary processing products form a non-covalent complex on the surface of merozoites (the invasive form of the parasite) during invasion. However, only MSP1(19) which is anchored to the merozoite membrane is carried into the newly infected erythrocyte while other fragments are shed into the surrounding medium.

In view of the foregoing, the invention provides isolated Band 3 peptides which selectively bind to merozoite surface protein-1 (MSP-1). The complete nucleic acid and amino acid sequences for human Band 3 protein are described in GenBank accession nos. X12609 and M27819 (SEQ ID NOs:5 and 6, (X12609 nucleic acid and amino acid sequences, respectively); and SEQ ID NOs:7 and 8 (M27819 nucleic acid and amino acid sequences, respectively); the complete amino acid and nucleic acid sequences for MSP-1 are described in GenBank accession no. X02919 (SEQ ID NOs:9 and 10, respectively). See also, (e.g., Fujinaga, J., et al. J Biol Chem 274:6626-6633, 1999) which reports the topology of the membrane domain of Band 3.

The results disclosed herein suggest that these particular Band 3 peptide sequences selectively interact with MSP-1 and, thereby, facilitate malaria parasite entry into erythrocytes. Such peptides are alternatively referred to herein as “Band 3 immunogenic polypeptides”, “Band 3-derived MSP-1 binding peptides”, and the like. Hence, one aspect of the invention is an isolated peptide selected from the group of sequences having SEQ ID Nos. 1, 2, 3, and 4, and unique fragments thereof which bind to MSP-1. The selection of these particular sequences and of the particular malaria protein with which they interact, could not have been predicted based on the information presently known regarding the Band 3 structure.

Although not wishing to be bound to any particular theory or mechanism, it is believed that P. falciparum depends on the expression on its surface of a specific molecule, MSP-1, also (referred to herein as a ligand), stereochemically complementary to the Band 3 receptor for its binding to and/or, subsequent penetration into the erythrocyte. This ligand can be inhibited in its binding to the receptor (in the specific case of P. falciparum, the receptor molecule corresponds to erythroid Band 3) by an antibody which selectively binds to SEQ ID NO. 1, 2, 3, and/or 4 (anti-Band 3 antibody) and which possesses the same sterochemical specificity. Thus, immunization with an anti-Band 3 antibody of the invention as a vaccine is useful for eliciting an immune response to its combining site, termed an anti-idiotypic response, resulting in antibodies which will also recognize the immunologically identical epitope on the ligand and, hence, will protect the host against the erythrocytic forms (merozoites) of P. falciparum. Thus, the present invention also involves the use of monoclonal anti-Band 3 antibodies immunochemically specific for the epitope used by P. falciparum to penetrate into host cells. These monoclonal antibodies are used according to the present invention, to generate or elicit the corresponding anti-idiotypic antibodies which, by virtue of their specificity for the parasite's ligand molecule (MSP-1) are useful in the serodiagnosis and treatment of established infection.

As used herein a “subject” shall mean a human, vertebrate, or invertebrate animal including but not limited to a dog, cat, horse, cow, pig, sheep, goat, non-human primate (e.g. monkey), rabbit, rat, mouse, avian, or insect (e.g. a mosquito). The malarial parasites of the invention include: P. falciparum, P. vivax, P. malariae, P. ovale, P. berghei, P. yoelii, P. chabaudi, P. vinckei, and P. knowlesi, P. cynomolgi, and P. coatneyi. A preferred malarial parasite of the invention is P. falciparum. As used herein, a “malarial infection” includes infection with a malarial parasite including: P. falciparum, P. vivax, P. malariae, P. ovale P. berghei, P. yoelii, P. chabaudi, P. vinckei, and P. knowlesi, P. cynomolgi, and/or P. coatneyi. A preferred malaria infection of the invention is infection with P. falciparum.

As used herein, the term “cell” means a cell capable of being infected by, or suspected of being exposed to a malarial parasite. This may include cells in or from a subject and cells grown in culture. A cell may also mean a cell collected from a subject such as a human or animal, for example, blood collected for purposes such as, but not limited to, transfusions. In some embodiments, a cell may be a negative control cell, which may be a cell that has not been exposed to a Plasmodium parasite. In some embodiments, a positive control cell may be a cell that has been exposed to a Plasmodium parasite but is free of a pharmaceutical agent of the invention. A cell is any cell that can be infected by a Plasmodium parasite, which includes, but is not limited to: mammalian cells, human cells, avian cells, insect cells, arthropod cells, neuronal cells, ocular cells, erythrocytes, lymphocytes, muscle cells, and intestinal cells.

One class of subjects according to the present invention are subjects having a Plasmodium infection. Such subjects are subjects in need of treatment with a Plasmodium inhibitor. This class of subjects includes subjects diagnosed with infection, exhibiting symptoms of infection, or having been exposed to a Plasmodium parasite. A subject at risk of developing a plasmodium infection is a subject in need of prevention of infection. Such subjects include those at risk of exposure to an infection-causing Plasmodium parasite. For instance, a subject at risk may be a subject who is planning to travel to an area where a particular type of infectious Plasmodium parasite is found or it may be a subject who through lifestyle, occupation, or medical procedures is exposed to bodily fluids which may contain a Plasmodium parasite or even any subject living in an area that a Plasmodium parasite has been identified. Subjects at risk of developing infection also include general populations to which a medical agency recommends preventative infectious measures for a particular infectious organism.

A subject may or may not exhibit symptoms of infection such as fever, swollen lymph glands, muscle aches, and pains. Methods to diagnose symptomatic and asymptomatic Plasmodium infection are known to those of ordinary skill in the medical arts and are described below herein. Some methods of diagnosis include, but are not limited to, blood tests for antibodies to the Plasmodium parasite and other assays such as lymph assays for Plasmodium parasites.

As noted above, the invention embraces functional variants, such as unique fragments, of the isolated Band 3 polypeptides of the invention which selectively bind to one or more of the peptides: MSP-1 (particularly SEQ ID NOS.: 11,12,13, 33, 34, 35), and the polypeptide molecules comprising an amino acid sequence selected from the group consisting of SEQ ID NOs:46-53. As used herein, a “functional variant” or “variant” of a Band 3 polypeptide of the invention is a molecule which contains one or more modifications to the primary amino acid sequence of the Band 3 polypeptide of the invention and retains the MSP-1 binding properties disclosed herein. Modifications which create a Band 3 immunogenic polypeptide functional variant can be made, for example, 1) to enhance a property of a Band 3 binding peptide, such as peptide stability in an expression system or the stability of protein-protein binding such as MSP-1 binding; or 2) to provide a novel activity or property to a Band 3 immunogenic polypeptide, such as addition of an antigenic epitope or addition of a detectable moiety. Modifications to a Band 3 polypeptide of the invention can be made to a nucleic acid which encodes the peptide, and can include deletions, point mutations, truncations, amino acid substitutions and additions of amino acids. Alternatively, modifications can be made directly to the polypeptide, such as by cleavage, addition of a linker molecule, addition of a detectable moiety, such as biotin, substitution of one amino acid for another and the like. Modifications also embrace fusion proteins comprising all or part of the Band 3 polypeptide amino acid sequence.

The amino acid sequence of Band 3 immunogenic polypeptides of the invention may be of natural or non-natural origin, that is, they may comprise a natural Band 3 polypeptide molecule or may comprise a modified sequence as long as the amino acid sequence retains the property of binding to MSP-1 and/or any of the other malaria polypeptides disclosed herein. For example, Band 3 polypeptides in this context may be fusion proteins of a Band 3 polypeptide of the invention and unrelated amino acid sequences, synthetic peptides of amino acid sequences shown in SEQ ID NOs: 1, 2, 3, and 4, peptides isolated from cultured cells which express Band 3 peptides, and peptides coupled to nonpeptide molecules (for example in certain drug delivery systems or detectable labels).

Nonpeptide analogs of the Band 3 peptides of the invention, e.g., those which provide a stabilized structure or lessened biodegradation, are also contemplated. Peptide mimetic analogs can be prepared based on a selected Band 3 peptide by replacement of one or more residues by nonpeptide moieties. Preferably, the nonpeptide moieties permit the peptide to retain its natural confirmation, or stabilize a preferred, e.g., bioactive, confirmation. One example of a method for preparation of nonpeptide mimetic analogs from peptides is described in Nachman et al., Regul. Pept. 57:359-370 (1995). Peptide mimetics also can be selected from libraries of synthetic compounds (e.g. combinatorial libraries of small organic molecules) or natural molecules according to the MSP-1 and/or other malaria polypeptide binding properties of such molecule (i.e., ability to selectively bind to MSP-1 and/or other malaria polypeptides disclosed herein (isolated or expressed on the surface of a cell or organism)) and/or inhibit parasite entry into human red blood cells. In general, the methods for selection involve determining whether the library's molecules inhibit selective binding of a Band 3 peptide of the invention (e.g., SEQ ID NOs: 1, 2, 3, or 4) to MSP-1 and/or to other malaria polypeptides disclosed herein and/or block the malaria parasite invasion of red blood cells by for example inhibiting a natural process which merozoites use to penetrate red blood cells.

If a variant involves a change to an amino acid of a Band 3 polypeptide (e.g., SEQ ID NOs: 1, 2, 3, or 4), functional variants of the Band 3 immunogenic polypeptide having conservative amino acid substitutions typically will be preferred, i.e., substitutions which retain a property of the original amino acid such as charge, hydrophobicity, conformation, etc. Examples of conservative substitutions of amino acids include substitutions made amongst amino acids within the following groups:

(a) M, I, L, V; (b) F, Y, W; (c) K, R, H; (d) A, G; (e) S, T; (f) Q, N; and (g) E, D.

Other methods for identifying functional variants of the Band 3 polypeptides rely upon the development of amino acid sequence motifs to which potential epitopes may be compared. (See, e.g., published PCT application of Strominger and Wucherpfennig (US/96/03182)). In general, these methods rely upon the development of amino acid sequence motifs to which potential epitopes may be compared. Each motif describes a finite set of amino acid sequences in which the residues at each (relative) position may be (a) restricted to a single residue, (b) allowed to vary amongst a restricted set of residues, or (c) allowed to vary amongst all possible residues. For example, a motif might specify that the residue at a Band 3 peptide position may be any one of the residues valine, leucine, isoleucine, methionine, or phenylalanine; that the residue at the second position must be histidine; that the residue at the third position may be any amino acid residue; that the residue at the fourth position may be any one of the residues valine, leucine, isoleucine, methionine, phenylalanine, tyrosine or tryptophan; and that the residue at the fifth position must be lysine.

Sequence motifs for the Band 3 peptide functional variants can be developed by analysis of the MSP-1 (or other malaria polypeptides of the invention) contact points of the Band 3 polypeptides disclosed herein. By providing a detailed structural analysis of the residues involved in the binding of the malaria polypeptides of the invention to the Band 3 polypeptides disclosed herein, one of ordinary skill in the art is enabled to make predictions of sequence motifs for binding between such pairs of proteins.

Using these sequence motifs as search, evaluation, or design criteria, one of ordinary skill in the art is enabled to identify classes of peptides (functional variants of the Band 3 peptides disclosed herein) which have a reasonable likelihood of binding to and of interacting with MSP-1 (or other malaria polypeptides of the invention) to inhibit parasite entry into erythrocytes. These peptides can be synthesized and tested for activity as described herein. Use of these motifs, as opposed to pure sequence homology (which excludes many peptides which are antigenically similar but quite distinct in sequence) or sequence homology with unlimited “conservative” substitutions (which admits many peptides which differ at critical highly conserved sites), represents a method by which one of ordinary skill in the art can evaluate peptides for potential application in the treatment of disease, such as malaria infection.

The binding of the variant Band 3 peptides to MSP-1 (or other malaria polypeptides of the invention) then is determined according to standard procedures. For example, the variant peptide can be contacted with the MSP-1 which binds the Band 3 peptides of the invention (e.g., SEQ ID NOs: 1, 2, 3, or 4) to form a complex of the variant peptide and MSP-1. This contacting can be performed in the presence of Band-3 expressed on erythrocytes to determine whether the variant peptide of Band 3 inhibits binding of the MSP-1 (e.g., expressed by a malaria parasite) and/or entry of the parasite into the erythrocyte.

Variant Band 3 peptides include “unique fragments” of the peptides having SEQ ID NOs: 1, 2, 3, and 4. As used herein, a unique fragment is one that is a ‘signature’ for the larger nucleic acid. It, for example, is long enough to assure that its precise sequence is not found in molecules within the human genome outside of the Band 3 nucleic acids defined above (and human alleles). Those of ordinary skill in the art may apply no more than routine procedures to determine if a fragment is unique within the human genome. Unique fragments, however, exclude fragments completely composed of the nucleotide sequences of any of GenBank accession numbers identified in Table 4 or other previously published sequences as of the filing date of the priority documents for sequences listed in a respective priority document or the filing date of this application for sequences listed for the first time in this application which overlap the sequences of the invention. Thus, unique Band 3 peptide fragments exclude the previously reported peptides identified in Table 4 (Blast results for SEQ ID NOs:1, 2, 3, or 4).

Binding of the variant peptide to the MSP-1 (or other malaria polypeptides of the invention) and/or blocking of the entry of MSP-1 (or other malaria polypeptides of the invention) (e.g., expressed by malaria parasite, containing a detectable label) into erythrocytes (or other cells expressing Band 3) indicates that the variant peptide is a functional variant. The methods also can include the step of comparing the blocking of Band 3-mediated MSP-1 (or other malaria polypeptides of the invention) (e.g., expressed on the merozoite surface) entry into erythrocytes by the Band 3 peptides or antibodies thereto (anti-Band 3 antibodies), as well as anti-idiotype antibodies, and the blocking by the functional variant as a determination of the effectiveness of the blocking by the functional variant. By comparing the functional variant with the Band 3 peptides or other compositions of the invention disclosed herein, peptides with increased Band 3 blocking properties can be prepared.

Variants of the Band 3 peptides prepared by any of the foregoing methods can be sequenced, if necessary, to determine the amino acid sequence and thus deduce the nucleotide sequence which encodes such variants. Thus, those nucleic acid sequences which code for a Band 3 peptide or variants thereof, including allelic variants, are also a part of the invention. In screening for nucleic acids which encode a Band 3 peptide of the invention, nucleic acid hybridization such as a Southern blot or a Northern blot may be performed under stringent conditions, together with a ³²P probe. The term “stringent conditions” as used herein refers to parameters with which the art is familiar. Nucleic acid hybridization parameters may be found in references which compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Exemplary stringent conditions include hybridization at 65° C. in hybridization buffer (3.5× SSC, 0.02% Ficoll, 0.02% Polyvinyl pyrolidone, 0.02% Bovine Serum Albumin, 25 mM NaH₂PO₄ (pH 7), 0.5% SDS, 2 mM EDTA). SSC is 0.15M Sodium Chloride/0.015M Sodium Citrate, pH 7; SDS is Sodium Dodecyl Sulphate; and EDTA is Ethylene diaminetetraacetic acid. After hybridization, the membrane upon which the DNA is transferred can be washed, for example, at 2× SSC at room temperature and then at 0.1-0.5× SSC/0.1× SDS at temperatures up to 68° C. After washing the membrane to which DNA encoding a Band 3 polypeptide is finally transferred, the membrane can be placed against X-ray film to detect the radioactive signal.

There are other conditions, reagents, and so forth which can be used, which result in a similar degree of stringency. The skilled artisan will be familiar with such conditions, and thus they are not given here. It will be understood, however, that the skilled artisan will be able to manipulate the conditions in a manner to permit the clear identification of homologs and alleles of nucleic acids encoding the Band 3 peptides of the invention. The skilled artisan also is familiar with the methodology for screening cells and libraries for expression of such molecules which then are routinely isolated, followed by isolation of the pertinent nucleic acid molecule and sequencing.

The invention also includes the use of nucleic acid sequences which include alternative codons that encode the same amino acid residues of the Band 3 peptides of the invention. For example, leucine residues can be encoded by the codons CUA, CUC, CUG, CUU, UUA and UUG. Each of the six codons is equivalent for the purposes of encoding a leucine residue. Thus, it will be apparent to one of ordinary skill in the art that any of the leucine-encoding nucleotide triplets may be employed to direct the protein synthesis apparatus, in vitro or in vivo, to incorporate a leucine residue. Similarly, nucleotide sequence triplets which encode other amino acid residues comprising the Band 3 peptides include: GUA, GUC, GUG and GUU (valine codons); GGU, GGA, GGG, GGC (glycine codons); UAC and UAU (tyrosine codons). Other amino acid residues may be encoded similarly by multiple nucleotide sequences. Thus, the invention embraces degenerate nucleic acids that differ from the native Band 3 peptide encoding nucleic acids in codon sequence due to the degeneracy of the genetic code.

Preferred nucleic acids encoding Band 3 polypeptides are those which preferentially express Band 3 peptides, such as those having SEQ ID NOs: 1, 2, 3, or 4. The Band 3 nucleic acids of the invention do not encode the entire Band 3 polypeptide but do include nucleotide sequences encoding the Band 3 peptides disclosed herein.

The invention also provides modified nucleic acid molecules which include additions, substitutions and deletions of one or more nucleotides. In preferred embodiments, these modified nucleic acid molecules and/or the polypeptides they encode retain at least one activity or function of the unmodified nucleic acid molecule and/or the polypeptides, such as antigenicity, ligand binding, formation of complexes by binding of peptides to MSP-1 (or other malaria polypeptides of the invention), etc. In certain embodiments, the modified nucleic acid molecules encode modified polypeptides, preferably polypeptides having conservative amino acid substitutions as are described elsewhere herein. The modified nucleic acid molecules are structurally related to the unmodified nucleic acid molecules and in preferred embodiments are sufficiently structurally related to the unmodified nucleic acid molecules so that the modified and unmodified nucleic acid molecules hybridize under stringent conditions known to one of skill in the art.

For example, modified nucleic acid molecules which encode polypeptides having single amino acid changes can be prepared. Each of these nucleic acid molecules can have one, two or three nucleotide substitutions exclusive of nucleotide changes corresponding to the degeneracy of the genetic code as described herein. Likewise, modified nucleic acid molecules which encode polypeptides having two amino acid changes can be prepared which have, e.g., 2-6 nucleotide changes. Numerous modified nucleic acid molecules like these will be readily envisioned by one of skill in the art, including for example, substitutions of nucleotides in codons encoding amino acids 2 and 3, 2 and 4, 2 and 5, 2 and 6, and so on. In the foregoing example, each combination of two amino acids is included in the set of modified nucleic acid molecules, as well as all nucleotide substitutions which code for the amino acid substitutions. Additional nucleic acid molecules that encode polypeptides having additional substitutions (i.e., 3 or more), additions or deletions (e.g., by introduction of a stop codon or a splice site(s)) also can be prepared and are embraced by the invention as readily envisioned by one of ordinary skill in the art. Any of the foregoing nucleic acids or polypeptides can be tested by routine experimentation for retention of structural relation or activity to the nucleic acids and/or polypeptides disclosed herein.

It will also be understood that the invention embraces the use of the sequences in expression vectors, as well as to transfect host cells and cell lines, be these prokaryotic (e.g., E. coli), or eukaryotic (e.g., CHO cells, COS cells, yeast expression systems and recombinant baculovirus expression in insect cells). The expression vectors require that the pertinent sequence, i.e., those described supra, be operably linked to a promoter.

Delivery of expression vectors encoding the Band 3 sequences in vivo and/or in vitro can be via the use of nucleic acid delivery systems known in the art (see, e.g., Allsopp et al., Eur. J. Immunol. 26(8):1951-1959, 1996). Recombinant vectors including viruses selected from the group consisting of adenoviruses, adeno-associated viruses, poxviruses including vaccinia viruses and attenuated poxviruses such as NYVAC, Semliki Forest virus, Venezuelan equine encephalitis virus, retroviruses, Sindbis virus, and Ty virus-like particle, plasmids (e.g. “naked” DNA), bacteria (e.g. the bacterium Bacille Calmette Guerin, BCG), and the like can be used in such delivery, for example, for use as a vaccine. Other viruses, expression vectors and the like which are useful in preparation of a vaccine are known to one of ordinary skill in the art. One can test the Band 3 delivery systems in standard model systems such as mice to determine efficacy of the delivery system. The systems also can be tested in human clinical trials.

As used herein, a “vector” may be any of a number of nucleic acids into which a desired sequence may be inserted by restriction and ligation for transport between different genetic environments or for expression in a host cell. Vectors are typically composed of DNA although RNA vectors are also available. Vectors include, but are not limited to, plasmids, phagemids, bacteria and virus genomes as disclosed herein, such as adenovirus, poxvirus and BCG. A cloning vector is one which is able to replicate in a host cell or be replicated after its integration into the genome of a host cell, and which is further characterized by one or more endonuclease restriction sites at which the vector may be cut in a determinable fashion and into which a desired DNA sequence may be ligated such that the new recombinant vector retains its ability to replicate in the host cell. In the case of plasmids, replication of the desired sequence may occur many times as the plasmid increases in copy number within the host bacterium or just a single time per host before the host reproduces by mitosis. In the case of phage, replication may occur actively during a lytic phase or passively during a lysogenic phase. An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase, luciferase or alkaline phosphatase), and genes which visibly affect the phenotype of transformed or transfected cells, hosts, colonies or plaques (e.g., green fluorescent protein). Preferred vectors are those capable of autonomous replication and expression of the structural gene products present in the DNA segments to which they are operably joined.

As used herein, a coding sequence and regulatory sequences are said to be “operably” joined when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript might be translated into the desired protein or polypeptide. As noted above, certain preferred nucleic acids express only fragments of Band 3 polypeptides which bind to the malaria peptides disclosed herein.

The precise nature of the regulatory sequences needed for gene expression may vary between species or cell types, but shall in general include, as necessary, 5′ non-transcribed and 5′ non-translated sequences involved with the initiation of transcription and translation respectively, such as a TATA box, capping sequence, CAAT sequence, and the like. Especially, such 5′ non-transcribed regulatory sequences will include a promoter region which includes a promoter sequence for transcriptional control of the operably joined gene. Regulatory sequences may also include enhancer sequences or upstream activator sequences as desired. The vectors of the invention may optionally include 5′ leader or signal sequences. The choice and design of an appropriate vector is within the ability and discretion of one of ordinary skill in the art.

Expression vectors containing all the necessary elements for expression are commercially available and known to those skilled in the art. See, e.g., Sambrook et al., Molecular Cloning: A Laboratory Manual, Second Edition, Cold Spring Harbor Laboratory Press, 1989. Cells are genetically engineered by the introduction into the cells of heterologous DNA (RNA) encoding a Band 3 peptide of the invention. That heterologous DNA (RNA) is placed under operable control of transcriptional elements to permit the expression of the heterologous DNA in the host cell.

Preferred systems for mRNA expression in mammalian cells are those such as pcDNA3.1 (available from Invitrogen, Carlsbad, Calif.) that contain a selectable marker such as a gene that confers G418 resistance (which facilitates the selection of stably transfected cell lines) and the human cytomegalovirus (CMV) enhancer-promoter sequences. Additionally, suitable for expression in primate or canine cell lines is the pCEP4 vector (Invitrogen), which contains an Epstein Barr virus (EBV) origin of replication, facilitating the maintenance of plasmid as a multicopy extrachromosomal element. Another expression vector is the pEF-BOS plasmid containing the promoter of polypeptide Elongation Factor 1α, which stimulates efficiently transcription in vitro. The plasmid is described by Mishizuma and Nagata (Nuc. Acids Res. 18:5322, 1990), and its use in transfection experiments is disclosed by, for example, Demoulin (Mol. Cell. Biol. 16:4710-4716, 1996). Still another preferred expression vector is an adenovirus, described by Stratford-Perricaudet, which is defective for E1 and E3 proteins (J. Clin. Invest. 90:626-630, 1992). The use of the adenovirus to express proteins for immunization is disclosed by Wamier et al., in intraderrnal injection in mice for immunization against P1A (Int. J. Cancer, 67:303-310, 1996).

The invention also embraces so-called expression kits, which allow the artisan to prepare a desired expression vector or vectors. Such expression kits include at least separate portions of at least two of the previously discussed materials. Other components may be added, as desired.

The invention further includes nucleic acid or protein microarrays which include Band 3 nucleic acids or peptides of the invention (preferably at least one isolated Band 3 peptide selected from the group consisting of SEQ ID NO: 1, 2, 3, or 4) or nucleic acids encoding such peptides. In this aspect of the invention, standard techniques of microarray technology are utilized to assess expression of the Band 3 binding peptides (e.g., anti-Band 3 antibodies) and/or identify biological constituents that bind such peptides. The constituents of biological samples include antibodies, MSP-1 molecules, other of the malaria peptide disclosed herein, and the like. Microarray technology, which is also known by other names including: protein chip technology and solid-phase protein array technology, is well known to those of ordinary skill in the art and is based on, but not limited to, obtaining an array of identified peptides or proteins on a fixed substrate, binding target molecules or biological constituents to the peptides, and evaluating such binding. See, e.g., G. MacBeath and S. L. Schreiber, “Printing Proteins as Microarrays for High-Throughput Function Determination,” Science 289(5485):1760-1763, 2000.

Microarray substrates include but are not limited to glass, silica, aluminosilicates, borosilicates, metal oxides such as alumina and nickel oxide, various clays, nitrocellulose, or nylon. The microarray substrates may be coated with a compound to enhance synthesis of a probe (peptide or nucleic acid) on the substrate. Coupling agents or groups on the substrate can be used to covalently link the first nucleotide or amino acid to the substrate. A variety of coupling agents or groups are known to those of skill in the art. Peptide or nucleic acid probes thus can be synthesized directly on the substrate in a predetermined grid. Alternatively, peptide or nucleic acid probes can be spotted on the substrate, and in such cases the substrate may be coated with a compound to enhance binding of the probe to the substrate. In these embodiments, presynthesized probes are applied to the substrate in a precise, predetermined volume and grid pattern, preferably utilizing a computer-controlled robot to apply probe to the substrate in a contact-printing manner or in a non-contact manner such as ink jet or piezo-electric delivery. Probes may be covalently linked to the substrate.

In some embodiments, one or more control peptide or nucleic acid molecules are attached to the substrate. Preferably, control nucleic acid molecules allow determination of factors such as binding characteristics, reagent quality and effectiveness, hybridization success, and analysis thresholds and success.

The present invention also concerns a general method for producing immunity against an infectious microorganism based on the stereochemical complementarity between the ligand molecule on the microorganism and its receptor in the host's cell. This complementarity is used according to the present invention to generate antibodies against the ligand by immunizing with monoclonal antibodies against the ligand-binding region of the receptor molecule. Such monoclonal anti-receptor antibodies, which react with the exact area of binding of the ligand molecule of the parasitic organism, bring about protective immunity because they elicit anti-idiotypic antibodies that react with the stereochemically equivalent region of the ligand.

The anti-Band 3 antibodies of the inventions selectively bind to SEQ ID NOs: 1, 2, 3, and/or 4 and, thereby block penetration of P. falciparum malaria parasite into human red blood cells by virtue of effectively blocking the site on the erythrocytic molecule (Band 3) used as a target by the P. falciparum malaria parasite. Accordingly, the invention provides monoclonal antibodies which have a combining site that has the same stereochemical configuration as the ligand site (e.g., MSP-1) on the P. falciparum malaria parasite. Such anti-Band 3 antibodies and anti-idiotpyic antibodies are prepared by standard methods. The selected cloned hybridomas produce as large quantities of suitable monoclonal antibodies as desired.

In view of the foregoing, the invention also permits the artisan to treat a subject having a malaria infection. Treatments include administering an anti-Band 3 binding peptide (e.g., an anti-Band 3 antibody) or other agent which inhibits binding of MSP-1 (or other malaria polypeptides of the invention) expressed by a malaria parasite to Band 3 expressed by an erythrocyte. Agents useful in the foregoing treatments include Band 3 polypeptides and functional variants thereof, as well as the anti-Band 3 antibodies and anti-idiotypic antibodies of the invention disclosed herein.

The function or status of a pharmaceutical agent as an Plasmodium inhibitor, can be determined according to assays known in the art or described herein. For example, cells can be contacted with a putative pharmaceutical agent and a Plasmodium parasite, and standard procedures can be used to determine whether the parasite is inhibited in its ability to enter or infect the cells. Such methods may also be utilized to determine the status of analogs, variants, derivatives, and fragments as inhibitors of invasion by Plasmodium parasites. One method for inhibiting infection is by inhibiting entry of Plasmodium parasite into cells. The ability to inhibit entry of Plasmodium parasite into cells with a putative pharmaceutical agent can be assessed using routine screening assays, e.g. by determining the level of entry of Plasmodium parasite into cells with and without the presence of the putative pharmaceutical agent.

Once the pharmaceutical agents are verified as modulating Plasmodium parasitic infection using secondary assays as described above herein, further biochemical and molecular techniques may be used to identify the targets of these compounds and to elucidate the specific roles that these target molecules play in the process of invasion. An example, though not intended to be limiting, is that the compound(s) may be labeled and contacted with a parasite to identify the host cell proteins with which these compounds interact. Such proteins may be purified, e.g., by labeling the compound with an immunoaffinity tag and applying the protein-bound compound to an immunoaffinity column.

In addition, the status of a pharmaceutical agent as a Plasmodium parasite toxin can be identified by using methods provided herein to determine the presence of a functional, active Plasmodium parasite. The agent may for example be assayed in the context of a material, for example a water sample, before and after contact with the sample and the pharmaceutical agent.

When administered, the therapeutic compositions of the present invention are administered in pharmaceutically acceptable preparations. Such preparations may routinely contain pharmaceutically acceptable concentrations of salt, buffering agents, preservatives, compatible carriers, and optionally other therapeutic agents.

The term “pharmaceutically acceptable” means a non-toxic material that does not interfere with the effectiveness of the biological activity of the active ingredients. The characteristics of the carrier will depend on the route of administration.

The therapeutics of the invention can be administered by any conventional route, including injection or by gradual infusion over time. The administration may, for example, be oral, intravenous, intraperitoneal, intramuscular, intranasal, intracavity, subcutaneous, intradermal, or transdermal.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

The preparations of the invention are administered in effective amounts. An effective amount is that amount of a pharmaceutical preparation that alone, or together with further doses, stimulates the desired response. In the case of treating an infectious disease such as a Plasmodium infection, the desired response is inhibiting the onset, stage or progression of the disease or infection. This may involve only slowing the progression of the disease temporarily, although more preferably, it involves halting the progression of the disease permanently. An effective amount for preventing infection is that amount that reduces the incidence of active infection when the cell or subject is exposed to the parasite, with respect to that amount that would occur in the absence of the active agent.

In another aspect of the invention, cell models and/or non-human animal models of Plasmodium infection may be produced by administering a molecule of the invention, such as an antibody, that inhibits Plasmodium infection. In some cases, a molecule of the invention that enhances Plasmodium invasion may be administered to an animal or cell. Such models may be useful for testing treatment strategies, monitoring clinical features of disease, or as tools to assess prevention strategies of Plasmodium infection. Cells and animal models made using enhancing molecules of the invention may also be useful for assessing the ability of lead compounds to inhibit Plasmodium infection. For example, a cell contacted with an enhancer of invasion of the invention may be further contacted with putative agents that are candidate or lead compounds for treating or preventing Plasmodium infection. The ability of the lead or candidate compound to prevent or treat the infection may be evaluated in the model cell or animal. In addition the enhancers may serve as valuable lead compounds in that if their targets (by definition functionally important) can be identified and characterized, it may subsequently be possible to rationally design new compounds that act as inhibitors of these targets. As used herein, an “effective amount of an enhancer” is that amount effective to enhance Plasmodium parasitic infection. Such enhancements can be determined using standard assays as described above herein. Measurements of Plasmodium parasitic infection, are known to those of ordinary skill in the art and may vary depending on the specific parasite.

The pharmaceutical compound dosage may be adjusted by the individual physician or veterinarian, particularly in the event of any complication. A therapeutically effective amount typically varies from 0.01 mg/kg to about 1000 mg/kg, preferably from about 0.1 mg/kg to about 200 mg/kg, and most preferably from about 0.2 mg/kg to about 20 mg/kg, in one or more dose administrations daily, for one or more days.

The absolute amount will depend upon a variety of factors, including the material selected for administration, whether the administration is in single or multiple doses, and individual patient parameters including age, physical condition, size, weight, and the stage of the disease. These factors are well known to those of ordinary skill in the art and can be addressed with no more than routine experimentation.

Diagnostic tests known to those of ordinary skill in the art may be used to assess Plasmodium infection status of a subject and to evaluate a therapeutically effective amount of a pharmaceutical agent administered. Examples of diagnostic tests are set forth below. A first determination of Plasmodium infection may be obtained using one of the methods described below (or other methods known in the art), and a subsequent determination of infection may be done. A comparison of the infection levels may be used to assess the effectiveness of administration of a pharmaceutical agent of the invention as a prophylactic or a treatment of the Plasmodium infection. Absence of a Plasmodium infection may be an indication for prophylactic intervention by administering a pharmaceutical agent described herein to prevent Plasmodium infection.

Tests useful for diagnosis of Plasmodium infections are known to those of ordinary skill in the art. For example, diagnosis of malaria can be done by microscopic identification of asexual forms of the parasite in peripheral blood smears stained with Romanovsky staining, or Giemsa at pH 7.2, Wright's, Field's, or Leishman's stain. Both thin and thick blood smears may be examined. In addition, a finger-prick blood test is also available, in which the presence of P. falciparum histidine-rich protein 2 is determined. Additional methods of diagnosis and assessment of Plasmodium infection are known to those of skill in the art. The level of parasitemia may be important in the prognosis and can be determined with the above-identified diagnostic tests and by other means known in the art.

In addition to the diagnostic tests described above, clinical features of Plasmodium infection can be monitored for assessment of infection. These features include, but are not limited to: normochromic, nomocytic anemia, erythrocyte sedimentation rate, plasma viscosity, and platelet count may be reduced. Subjects may also have metabolic acidosis, with low plasma concentrations of glucose, sodium, bicarbonate, calcium, phosphate, and albumin together with elevations in lactate, blood urea nitrogen, creatinine, urate, muscle and liver enzymes, and conjugated and unconjugated bilirubin. In adults and children with cerebral malaria, the mean opening pressure at lumbar puncture is about 160 mm cerebrospinal fluid; the cerebrospinal fluid usually is normal or has a slightly elevated total protein level [<1.0 g/L (100 mg/dL)] (see Harrison's Principles of Internal Medicine, 14/e, McGraw Hill Companies, New York, 1998).

The identification of Plasmodium parasites in or on an object, may be performed via standard diagnostic methods described above including microscopic examination, antibody labeling in a sample of the object, and by PCR analysis of a sample.

The pharmaceutical agents of the invention may be administered alone, in combination with each other, and/or in combination with other anti-Plasmodium drug therapies. Anti-malarial agents (for treatment and/or prophylaxis) that may be administered with pharmaceutical agents of the invention include, but are not limited to: mefloquine, doxycycline, chloroquine, aminoquinolines, dihydrofolate reductase inhibitors: pyrimethamine and proguanil (chloroguanide), dapsone, quinidine gluconate, quinine, artemisinin derivatives: artemether and artesunate, and primaquin. Methods of anti-plasmodium treatment of the invention may also be used in combination with drugs that target sialic acid interactions.

The above-described drug therapies are known to those of ordinary skill in the art and are administered by modes known to those of skill in the art. The drug therapies are administered in amounts that are effective to achieve the physiological goals (to reduce Plasmodium infection, and/or reduce Plasmodium parasite titer in a subject), in combination with the pharmaceutical agents of the invention. Thus, it is contemplated that the drug therapies may be administered in amounts which are not capable of preventing or reducing the physiological consequences of the Plasmodium infections when the drug therapies are administered alone, but which are capable of preventing or reducing the physiological consequences of Plasmodium infection when administered in combination with the pharmaceutical agents of the invention.

The pharmaceutical agents of the invention may also be administered in conjunction with vaccine formulations administered to confer immunity to a subject at risk of exposure to Plasmodium infection, which thereby prevents, reduces the severity of, or delays the onset of a subsequent Plasmodium infection.

The invention also provides a pharmaceutical kit comprising one or more containers comprising one or more of the pharmaceutical agents of the invention and or formulations of the invention. The kit may also include instructions for the use of the one or more phannaceutical agents or formulations of the invention for the treatment of Plasmodium infection.

In other aspects the invention involves preventing and/or treating Plasmodium contamination of materials. A “material” as used herein is any liquid or solid material including, but not limited to: blood, tissue, bodily fluids, and tissue-processing equipment, including but not limited to: equipment for food processing, medical equipment, equipment for tissue transplant processing, and equipment for cell or bodily fluid processing. In some embodiments of the invention, the material is aqueous. In some embodiments, the material is water, an example of which, although not intended to be limiting, is drinking water. The invention also involves preventing and/or treating Plasmodium contamination in blood, bodily fluids, cells, and tissue samples, including those from live human subjects and cadavers, as well as live animals and animal tissues and cells processed as food, cosmetics, or medication. As used herein, the term “contamination” means contact between the material and a Plasmodium parasite.

The isolated Band 3 peptides or anti-Band 3 antibodies of the invention may be combined with materials such as adjuvants to produce vaccines to prepare, respectively, anti-Band 3 antibodies that selectively bind to the portion of Band 3 involved in binding to MSP-1 and to prepare anti-idiotype antibodies that selectively bind to the portion of MSP-1 involved in binding to Band 3. Vaccines also encompass expression vectors and naked DNA or RNA, encoding a Band 3 peptide or anti-Band 3 antibody of the invention, precursors thereof, or fusion proteins thereof, which may be produced in vitro and administered via injection, particle bombardment, nasal aspiration and other methods. Vaccines of the “naked nucleic acid” type have been demonstrated to provoke an immunological response including generation of CTLs specific for the peptide encoded by the naked nucleic acid (Science 259:1745-1748, 1993).

In certain embodiments, the Band 3 peptides and anti-Band 3 antibodies of the invention are used to produce antibodies (“anti-Band 3 antibodies”) which, in turn, may be used to produce “anti-idiotypic Band 3 antibodies”, using standard techniques well known to the art. Standard reference works setting forth the general principles of antibody production include Catty, D., Antibodies, A Practical Approach, Vol. 1, IRL Press, Washington D.C. (1988); Klein, J., Immunology: The Science of Cell-Non-Cell Discrimination, John Wiley and Sons, New York (1982); Kennett, R., et al., Monoclonal Antibodies, Hybridoma, A New Dimension In Biological Analyses, Plenum Press, New York (1980); Campbell, A., Monoclonal Antibody Technology, in Laboratory Techniques and Biochemistry and Molecular Biology, Vol. 13 (Burdon, R. et al. EDS.), Elsevier Amsterdam (1984); and Eisen, H. N., Microbiology, third edition, Davis, B. D. et al. EDS. (Harper & Rowe, Philadelphia (1980). See also, U.S. Pat. No. 5,101,017, issued Mar. 31, 1992 to Rubinstein, et al., entitled, “Antibodies for providing protein against P. vivax malaria infection,” which also reports the preparation of anti-idiotypic antibodies for treating infectious disease.

References which report vaccine approaches for treating malaria include: U.S. Pat. No. 6,066,623, issued to Hoffman, et al., entitled “Polynucleotide vaccine protective against malaria, methods of protection and vector for delivering polynucleotide vaccines”; and U.S. Pat. No. 6,120,770, issued to Adams et al., entitled “Plasmodium proteins useful for preparing vaccine compositions.” Additional references which report anti-idiotype vaccines for treating various disorders include: Bendandi, Leukemia, 2000, 14(8): 1333-9; Bhattacharya-Chatterjee et al., Immunol. Lett., 2000,15:74(1):51-8; Maruyama et al., Cancer Immunol. Immunother., 2000, 49(3):123-32; Herlyn et al., Exp. Clin. Immunogenet., 1988, 5(4):165-75; Finberg et al., Crit. Rev. Immunol. 1987, 7(4):269-84; Nisonoff, American Association of Immunologists, 1991, 147(8):2429-2438; Greenspan et al., FASEB J., 1993, 7:437-444; and Syrengelas et al., The Journal of Immunology, 1999, 162:4790-4795.

Thus, according to one aspect of the invention, an anti-Band 3 antibody (or fragment thereof) that selectively binds to a peptide having SEQ ID NO: 1, 2, 3, and/or 4 and which blocks penetration of P. falciparum merozoite malaria parasite into human red blood cells is provided. According to yet another aspect of the invention, an anti-idiotype antibody which selectively binds to the idiotype of the anti-Band 3 antibodies described herein is provided. The antibodies of the present invention are prepared using any of a variety of methods, including administering the Band 3 peptides of the invention, fragments of the foregoing, antibodies selective for the foregoing, and the like to an animal to induce monoclonal or polyclonal antibodies. The production of monoclonal antibodies is according to techniques well known in the art.

Since the antibodies selective for the Band 3 epitope(s) (herein, anti-Band 3 antibodies) and the malaria polypeptide ligand(s) on the parasite recognize the same epitope, antibodies specific for the combining site on the anti-Band 3 antibodies (anti-idiotypic antibodies), can be elicited, which will react with the parasite's ligand(s). Susceptible individuals who make these anti-idiotypic antibodies will be protected against P. falciparum merozoites because (a) they block the ability of the parasite to recognize the erythrocytic receptor and (b) they may induce the lysis or inactivation of the Plasmodium cell by fixing complement.

The monoclonal anti-Band 3 antibodies according to the invention have the following characteristics and properties:

(1) The antibodies can be used as the immunogenic agent in a vaccine and can be produced in virtually limitless quantity.

(2) The region of the antibody molecule that bears the immunogenic moiety (idiotype) is located in the variable region which may be further purified in order to avoid the undesired immunogenicity of the constant region of the molecule.

(3) If desired, the antigen combining region of the antibody can be transferred to a carrier molecule devoid of additional immunogenic properties for human subjects. As discussed below, this may be done by a number of methods that are equivalent in this regard, such as, by chemically binding the Fab fragment of the antibody molecule to an Fc fragment derived from human immunoglobulin or by genetically engineering an appropriate hybrid molecule using the necessary portion of the rearranged immunoglobulin heavy and light chain genes from the monoclonal-producing hybridoma cell line into human immunoglobulin genes from which the equivalent regions have been excised. Alternatively, the idiotype-bearing portions of the protein (or the DNA fragments encoding them) may be attached to other immunogenic molecules or particles (or to their respective genetic determinants in the case of the DNA fragments encoding the idiotypes).

(4) The Band 3 peptides of the invention may be used for binding to and isolating the parasite's specific recognition molecule(s) (e.g., MSP-1). The purified ligand molecule(s) from the merozoite can then be characterized and used as “blue-prints” for the preparation of synthetic peptides (Band 3 functional variants) with protective immunogenic properties.

(5) The antibodies can be used to prepare anti-idiotypic monoclonal antibodies in mice. Those anti-idiotypic antibodies that additionally react with the combining site of P. falciparum merozoites can be used as affinity probes, to isolate the ligand as is described in (4) above for the receptor on red blood cells and with the same objectives.

(6) The antibodies and the anti-idiotypic antibody can be used in the immunodiagnosis of P. falciparum infection. Thus, the presence of P. falciparum antigen in serum or other fluid may be detected and its concentration measured by its interference with the binding of the monoclonal anti-Band 3 to either the Band 3 molecule or to its monoclonal anti-idiotypic antibody. Since the parasite's ligand(s) and the anti-Band 3 antibodies will react with the same respective combining site both on the Band 3 molecule and in the monoclonal anti-idiotypic immunoglobulin, a simple competition assay can be designed using either enzyme-linked or radiolabeled reagents, or other labeling reagents.

(7) The anti-Band 3 antibodies may be used directly in vivo to block the red cell receptors for the parasite. This might be useful in the management of patients with particularly severe attacks of P. falciparum malaria, in whom the level of parasitemia may be very high. In the same type of patients, but not simultaneously, passively administered anti-idiotypic antibodies may be useful by directly binding to and destroying the parasites.

The present invention also provides a method for the detection of the presence of P. falciparum infection in a patient. The method employs insolubilized monoclonal antibody which identifies Band 3 and labeled, e.g., radiolabeled or enzyme labeled, monoclonal anti-idiotypic antibody to the aforesaid monoclonal antibody. Soluble P. falciparum in the test sample will interfere with the binding to the insolublized monoclonal antibody of the labeled monoclonal anti-idiotypic antibody and will thus decrease the amount of the detectable label, e.g., the radioactivity or the enzyme, bound by the insolublized antibody.

Non-limiting examples of supports for affinity-separation of antibodies, including monoclonals, include the following: activated Sepharose, activated cellulose and activated Sephadex. “Activated” refers to the creation, on the insoluble material, of reactive chemical groups that will form covalent linkages with the antibody molecules when incubated together under appropriate conditions. Typically, reactive groups are introduced into the insoluble substrate by the action of cyanogen bromide (CNBr) at high pH.

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The pFc′ and Fc regions, for example, are effectors of the complement cascade but are not involved in antigen binding. An antibody from which the pFc′ region has been enzymatically cleaved, or which has been produced without the pFc′ region, designated an F(ab′)₂ fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, there are complementary determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDR3). The CDRs, and in particular the CDR3 regions, and more particularly the heavy chain CDR3, are largely responsible for antibody specificity.

It is now well-established in the art that the non-CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody. See, e.g., U.S. Pat. Nos. 4,816,567, 5,225,539, 5,585,089, 5,693,762 and 5,859,205. Accordingly, humanized anti-Band 3 antibodies and the use of such antibodies (e.g., to provide passive immunity to a subject) are embraced with the inventions disclosed herein.

For example, PCT International Publication Number WO 92/04381 teaches the production and use of humanized murine RSV antibodies in which at least a portion of the murine FR regions have been replaced by FR regions of human origin. Such antibodies, including fragments of intact antibodies with antigen-binding ability, are often referred to as “chimeric” antibodies.

Thus, the present invention also provides for F(ab′)₂, Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)₂ fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies and human monoclonal antibodies, such as those produced by mice having functional human immunoglobulin gene loci.

Such antibodies also may be used to identify tissues expressing protein or to purify protein. Antibodies, particularly the anti-idiotypic antibodies of the invention, also may be coupled to specific labeling agents for imaging or to anti-infectious agents, toxins such as ricin, other cytostatic or cytolytic drugs, and so forth, for therapeutic purposes.

As part of certain immunization compositions, one or more anti-Band 3 antibodies or stimulatory fragments thereof are administered with one or more adjuvants to induce an immune response or to increase an immune response. An adjuvant is a substance incorporated into or administered with antigen which potentiates the immune response. Adjuvants may enhance the immunological response by providing a reservoir of antigen (extracellularly or within macrophages), activating macrophages and stimulating specific sets of lymphocytes. Adjuvants of many kinds are well known in the art. Specific examples of adjuvants include monophosphoryl lipid A (MPL, SmithKline Beecham), a congener obtained after purification and acid hydrolysis of Salmonella minnesota Re 595 lipopolysaccharide; saponins including QS21 (SmithKline Beecham), a pure QA-21 saponin purified from Quillaja saponaria extract; DQS21, described in PCT application WO96/33739 (SmithKline Beecham); QS-7, QS-17, QS-18, and QS-L1 (So et al., Mol. Cells 7:178-186, 1997); incomplete Freund's adjuvant; complete Freund's adjuvant; montanide; immunostimulatory oligonucleotides (see e.g. CpG oligonucleotides described by Kreig et al., Nature 374:546-9, 1995); and various water-in-oil emulsions prepared from biodegradable oils such as squalene and/or tocopherol. Other adjuvants are known in the art and can be used in the invention (see, e.g. Goding, Monoclonal Antibodies: Principles and Practice, 2nd Ed., 1986). Methods for the preparation of mixtures or emulsions of peptide and adjuvant are well known to those of skill in the art of vaccination.

Other agents which stimulate the immune response of the subject can also be administered to the subject. For example, other cytokines are also useful in vaccination protocols as a result of their lymphocyte regulatory properties. Many other cytokines useful for such purposes will be known to one of ordinary skill in the art, including interleukin-12 (IL-12) which has been shown to enhance the protective effects of vaccines (see, e.g., Science 268: 1432-1434, 1995), GM-CSF and IL-18. Thus cytokines can be administered in conjunction with antigens and adjuvants to increase the immune response to the antigens.

There are a number of additional immune response potentiating compounds that can be used in vaccination protocols. These include costimulatory molecules provided in either protein or nucleic acid form. Such costimulatory molecules include the B7-1 and B7-2 (CD80 and CD86 respectively) molecules which are expressed on dendritic cells (DC) and interact with the CD28 molecule expressed on the T cell. This interaction provides costimulation (signal 2) to an antigen/MHC/TCR stimulated (signal 1) T cell, increasing T cell proliferation and effector function. B7 also interacts with CTLA4 (CD152) on T cells and studies involving CTLA4 and B7 ligands indicate that the B7-CTLA4 interaction can enhance antitumor immunity and CTL proliferation (Zheng et al., Proc. Nat'l Acad. Sci. USA 95:6284-6289, 1998).

The invention also contemplates gene therapy. The procedure for performing ex vivo gene therapy is outlined in U.S. Pat. No. 5,399,346 and in exhibits submitted in the file history of that patent, all of which are publicly available documents. In general, it involves introduction in vitro of a functional copy of a gene into a cell(s) of a subject which contains a defective copy of the gene, and returning the genetically engineered cell(s) to the subject. The functional copy of the gene is under operable control of regulatory elements which permit expression of the gene in the genetically engineered cell(s). Numerous transfection and transduction techniques as well as appropriate expression vectors are well known to those of ordinary skill in the art, some of which are described in PCT application WO95/00654. In vivo gene therapy using vectors such as adenovirus also is contemplated according to the invention.

Where it is desired to stimulate an immune response using a therapeutic composition of the invention, this may involve the stimulation of a humoral antibody response resulting in an increase in antibody titer in serum, a clonal expansion of cytotoxic lymphocytes, or some other desirable immunologic response. It is believed that doses of immunogens ranging from one nanogram/kilogram to 100 milligrams/kilogram, depending upon the mode of administration, would be effective. The preferred range is believed to be between 500 nanograms and 500 micrograms per kilogram. The absolute amount will depend upon a variety of factors, including the material selected for administration, whether the administration is in single or multiple doses, and individual patient parameters including age, physical condition, size, weight, and the stage of the disease. These factors are well known to those of ordinary skill in the art and can be addressed with no more than routine experimentation.

In addition to the above-described inventions which are based, in part, on the discovery of the particular Band 3 sequences which interact with MSP1 (or other malaria polypeptides of the invention), Applicants disclose herein related inventions which are based, in part, on the discovery of the particular portions of MSP1 (or other malaria polypeptides of the invention) that selectively binds to Band 3 protein. It is to be understood that enablement of the MSP1 (or other malaria polypeptides of the invention) related inventions throughout a broad scope can be accomplished in an analogous manner to that described for the Band 3 protein-related invention. Thus, the methods and definitions applied above in reference to the Band 3 molecule compositions and methods can be used in reference to the MSP1 molecule (or other malaria polypeptides of the invention) compositions and methods by substituting the MSP1 molecule (or other malaria polypeptides of the invention) for the Band 3 molecule. For example, vectors expressing an MSP1 protein can be prepared by substituting an MSP1 nucleic acid for a Band 3 nucleic acid and inserting into an expression vector as described above. Thus, the invention embraces various compositions containing such MSP-1 peptides for use, e.g., in screening assays to detect the specific interaction between MSP-1 and Band 3 protein, as well as for use in diagnostic and therapeutic applications for detecting and treating, respectively, malaria infection. In general, such compositions contain components (or are contained in kits which contain additional components) which are selected to detect the specific interaction between MSP-1 (or other malaria polypeptides of the invention) and Band 3 protein (particularly, the Band 3 peptides disclosed herein).

According to one aspect of the invention, an isolated MSP1 peptide is provided. The peptide has an amino acid sequence selected from the group consisting of (Wellcome strain) SEQ ID NO: 11 (MSP1(42)), SEQ ID NO: 12 (MSP1(38), and SEQ ID NO: 13 (MSP1(38)-N-terminal domain) and (FCB1 strain) MSP1₃₈ (SEQ ID NO:33), MSP1₄₂ (SEQ ID NO:34), and MSP1₁₉ (SEQ ID NO:35), and unique fragments thereof which bind Band 3 protein. The sequences for peptides SEQ ID NOs: 11-13 are based on P. falciparum Wellcome strain: GenBank Accession no.X02919 as follows:

SEQ ID NO:11 (MSP1(42)) refers to X02919 amino acids 1262 to 1639, inclusive;

SEQ ID NO:12 (MSP1(38)) refers to X02919 amino acids 902 to 1261, inclusive; and

SEQ ID NO: 13 (MSP1(38)—N terminal region) refers to X02919 amino acids 902 to 1121, inclusive.

The sequences for peptides SEQ ID Nos:33-35 are based on P. falciparum FCB1 strain. The sequence of polypeptide SEQ ID NO:33 is deposited as GenBank Accession No.:AF286876, amino acids 911-1263. The sequence for polypeptide SEQ ID NO:34 is deposited as GenBank Accession No: AF325919; amino acids 1264-1639. The sequence for polypeptide SEQ ID NO:35 is the C-terminal domain of MSP1(42); amino acids 1526-1639.

Such peptides may be contained in kits which detect the selective binding of the MSP1 peptide to a Band 3 protein, particularly to the Band 3 peptides disclosed herein.

According to another aspect of the invention, isolated nucleic acids encoding the foregoing MSP1 (or other malaria polypeptides of the invention) peptides are provided. The MSP1 nucleic acids have nucleotide sequences selected from the group consisting of (Wellcome strain) SEQ ID NO:54 (MSP1(42)), SEQ ID NO:55 (MSP1(38), and SEQ ID NO:56 (MSP1(38)-N-terminal domain) and (FCB1 strain) MSP1₃₈ (SEQ ID NO:57), MSP1₄₂ (SEQ ID NO:58), and MSP1₁₉ (SEQ ID NO:59), and unique fragments thereof. The sequences for nucleotide SEQ ID NOs:54-56 are based on P. falciparum Wellcome strain: GenBank Accession no.X02919. The sequences for nucleic acids SEQ ID Nos:57-59 are based on P. falciparum FCB1 strain, and are included in the nucleic acid sequences deposited as GenBank Accession Nos. AF286876 and AF325919

In certain embodiments, the nucleic acids comprise a unique fragment of the nucleotide sequence encoding the MSP1 peptides (or other malaria polypeptides of the invention) that selectively bind to Band 3. It is to be understood that all of the definitions described above in reference to the Band 3 molecules also apply to the MSP1 molecules (or other malaria polypeptides of the invention) of the invention. Thus, for example, the same definitions described above for “unique fragment”, “isolated”, “expression vectors”, “alternate codons”, and “operably joined” in reference to the Band 3 molecules of the invention apply to the MSP1 molecules (or other malaria polypeptides of the invention) of the invention. The expression vectors include the isolated foregoing MSP1 (or other malaria polypeptides of the invention) nucleic acids operably linked to a promoter. In another aspect of the invention, host cells transfected or transformed with the foregoing MSP1 (or other malaria polypeptides of the invention) nucleic acids or expression vectors are provided.

According to another aspect of the invention, compositions comprising the foregoing MSP1 (or other malaria polypeptides of the invention) peptides or nucleic acids and methods for making same are provided. The compositions are useful for inducing an immune response and include, e.g., immunogenic compositions. In general, the immunogenic compositions of the invention comprise one or more of the foregoing isolated MSP1 (or other malaria polypeptides of the invention) polypeptides, a pharmaceutically acceptable carrier; and, optionally, an adjuvant, wherein the polypeptides are present in the composition in an effective amount to induce an immune system response. Antibodies to MSP1 peptides can be used, for example, in kits to identify agents which competitively bind to MSP1 and inhibit MSP1 binding to Band 3 protein or peptides as disclosed herein. According to a related aspect, a method of making a medicament (including, e.g., an immunogenic composition) is provided. The method involves placing one or more of the foregoing isolated MSP1 (or other malaria polypeptides of the invention) polypeptides or nucleic acids in a pharmaceutically acceptable carrier to form the medicament.

According to yet another aspect of the invention, a protein microarray comprising at least one isolated MSP-1 peptide selected from the group consisting of SEQ ID NOS. 11, 12, 13, 33, 34, and 35 is provided. Alternatively, a nucleic acid microarray comprising at least one nucleic acid encoding any of the foregoing MSP-1 peptides is provided. The arrays are useful in identifying agents which selectively bind to MSP1 molecules and inhibit MSP1 binding to Band 3 protein or peptides as disclosed herein.

According to still another embodiment, a method for identifying a candidate mimetic of an isolated MSP1 peptide is provided. The method involves: providing a Band 3 molecule (e.g., the above-described isolated Band 3 peptides) which binds an isolated MSP1 peptide (e.g., an MSP1 peptide having SEQ ID NOs: 11, 12, 13, 33, 34, and/or 35); contacting the Band 3 molecule with the MSP1 peptide in the presence or absence of a test molecule; and determining the binding of the test molecule to the Band 3 molecule, wherein a test molecule which binds to the Band 3 molecule and inhibits binding of the Band 3 molecule with the MSP1 peptide is a candidate mimetic of the isolated peptide.

According to still another aspect of the invention, an antibody (or fragment thereof) (referred to herein as a “anti-MSP1 antibody”) that: (1) selectively binds to any of the above-described peptides, particularly the peptides having a sequence selected from the group consisting of SEQ ID Nos. 11, 12, 13, 33, 34, and 35; and (2) inhibits binding of the MSP1 peptide to Band 3 protein (particularly the Band 3 peptides disclosed herein) is provided. Preferably, the antibody is a monoclonal antibody, and, more preferably, a humanized monoclonal antibody.

In addition to the inventions which are based, in part, on the discovery of the particular Band 3 sequences which interact with MSP1, Applicants disclose herein related inventions which are based, in part, on the discovery that Band 3 sequences interact with additional Plasmodium polypeptide sequences comprising the amino acid sequences selected from the group consisting of SEQ ID NOs:46-53. These polypeptides are encoded by the nucleic acid molecules comprising a nucleotide sequence selected from the group consisting of SEQ ID NOs:38-45. It is to be understood that enablement of the BBP-1, BBP-2, BBP-3, BBP-4, BBP-5, BBP-6, RhopH3, and ABRA-related inventions throughout a broad scope can be accomplished in an analogous manner to that described for the Band 3 protein-related invention and the MSP1 polypeptide-related invention. Thus, the methods and definitions applied above in reference to the Band 3 and MSP-1 molecule compositions and methods can be used in reference to the BBP-1, BBP-2, BBP-3, BBP-4, BBP-5, BBP-6, RhopH3, and/or ABRA molecule compositions and methods by substituting the BBP-1, BBP-2, BBP-3, BBP-4, BBP-5, BBP-6, RhopH3, and/or ABRA molecule for the MSP-1 molecule. For example, vectors expressing an BBP-1 polypeptide can be prepared by substituting a BBP-1 nucleic acid for a MSP-1 nucleic acid and inserting into an expression vector as described above. Thus, the invention embraces various compositions containing such BBP-1, BBP-2, BBP-3, BBP-4, BBP-5, BBP-6, RhopH3, and/or ABRA polypeptides for use, e.g., in screening assays to detect the specific interaction between BBP-1, BBP-2, BBP-3, BBP-4, BBP-5, BBP-6, RhopH3, and/or ABRA and Band 3 protein, as well as for use in diagnostic (e.g. detection of the malaria polypeptides of the invention in biological samples obtained from a subject using conventional assays, such as an antibody-based assays or nucleic acid hybridization-based assays) and therapeutic applications (e.g., vaccines) for detecting and treating, respectively, malaria infection. The sequence identification for these polypeptides is provided in Table 1. In general, such compositions contain components (or are contained in kits which contain additional components) which are selected to detect the specific interaction between BBP-1, BBP-2, BBP-3, BBP-4, BBP-5, BBP-6, RhopH3, and/or ABRA, and Band 3 protein (particularly, the Band 3 peptides disclosed herein).

TABLE 1
Band 3 Binding Polypeptide Identification

| | Nucleic Acid | Amino Acid |
|---|---|---|
| Polypeptide Name | SEQ ID NO. | SEQ ID NO. |
| BBP-1 | 38 | 46 |
| BBP-2 | 39 | 47 |
| BBP-3 | 40 | 48 |
| BBP-4 | 41 | 49 |
| BBP-5 | 42 | 50 |
| BBP-6 | 43 | 51 |
| RhopH3 | 44 | 52 |
| ABRA | 45 | 53 |

According to one aspect of the invention, an isolated BBP-1, BBP-2, BBP-3, BBP-4, BBP-5, BBP-6, RhopH3, and/or ABRA peptide is provided. The peptide has an amino acid sequence selected from the group consisting of SEQ ID NOs:46-53 and unique fragments thereof that bind Band 3 protein. The sequences for these peptides are presented herein.

Such peptides may be contained in kits which detect the selective binding of the BBP-1, BBP-2, BBP-3, BBP-4, BBP-5, BBP-6, RhopH3, and/or ABRA polypeptide to a Band 3 protein, particularly to the Band 3 peptides disclosed herein.

According to another aspect of the invention, isolated nucleic acids encoding the foregoing BBP-1, BBP-2, BBP-3, BBP-4, BBP-5, BBP-6, RhopH3, and/or ABRA polypeptides are provided. In certain embodiments, the nucleic acids comprise a unique fragment of the nucleotide sequence encoding the BBP-1, BBP-2, BBP-3, BBP-4, BBP-5, BBP-6, RhopH3, and/or ABRA polypeptides that selectively bind to Band 3 peptides. It is to be understood that all of the definitions described above in reference to the Band 3 and MSP-1 molecules also apply to the BBP-1, BBP-2, BBP-3, BBP-4, BBP-5, BBP-6, RhopH3, and/or ABRA molecules of the invention. Thus, for example, the same definitions described above for “unique fragment”, “isolated”, “expression vectors”, “alternate codons”, and “operably joined” in reference to the Band 3 and MSP-1 molecules of the invention apply to the BBP-1, BBP-2, BBP-3, BBP-4, BBP-5, BBP-6, RhopH3, and/or ABRA molecules of the invention. The expression vectors include the isolated foregoing BBP-1, BBP-2, BBP-3, BBP-4, BBP-5, BBP-6, RhopH3, and/or ABRA nucleic acids operably linked to a promoter. In another aspect of the invention, host cells transfected or transformed with the foregoing BBP-1, BBP-2, BBP-3, BBP-4, BBP-5, BBP-6, RhopH3, and/or ABRA nucleic acids or expression vectors are provided.

According to another aspect of the invention, compositions comprising the foregoing BBP-1, BBP-2, BBP-3, BBP-4, BBP-5, BBP-6, RhopH3, and/or ABRA polypeptides or nucleic acids and methods for making same are provided. The compositions are useful for inducing an immune response and include, e.g., immunogenic compositions. In general, the immunogenic compositions of the invention comprise one or more of the foregoing isolated BBP-1, BBP-2, BBP-3, BBP-4, BBP-5, BBP-6, RhopH3, and ABRA polypeptides, a pharmaceutically acceptable carrier; and, optionally, an adjuvant, wherein the polypeptides are present in the composition in an effective amount to induce an immune system response. Antibodies to BBP-1, BBP-2, BBP-3, BBP-4, BBP-5, BBP-6, RhopH3, and ABRA polypeptides can be used, for example, in kits to identify agents which competitively bind to BBP-1, BBP-2, BBP-3, BBP-4, BBP-5, BBP-6, RhopH3, and/or ABRA and inhibit BBP-1, BBP-2, BBP-3, BBP-4, BBP-5, BBP-6, RhopH3, and/or ABRA binding to Band 3 protein or peptides as disclosed herein. According to a related aspect, a method of making a medicament (including, e.g., an immunogenic composition) is provided. The method involves placing one or more of the foregoing isolated BBP-1, BBP-2, BBP-3, BBP-4, BBP-5, BBP-6, RhopH3, and/or ABRA polypeptides or nucleic acids in a pharmaceutically acceptable carrier to form the medicament.

According to yet another aspect of the invention, a protein microarray comprising at least one isolated BBP-1, BBP-2, BBP-3, BBP-4, BBP-5, BBP-6, RhopH3, and/or ABRA polypeptide selected from the group consisting of SEQ ID NOs:46-53 is provided. Alternatively, a nucleic acid microarray comprising at least one nucleic acid encoding any of the foregoing BBP-1, BBP-2, BBP-3, BBP-4, BBP-5, BBP-6, RhopH3, and/or ABRA polypeptides is provided. The arrays are useful in identifying agents which selectively bind to BBP-1, BBP-2, BBP-3, BBP-4, BBP-5, BBP-6, RhopH3, and/or ABRA molecules and inhibit BBP-1, BBP-2, BBP-3, BBP-4, BBP-5, BBP-6, RhopH3, and/or ABRA binding to Band 3 protein or peptides as disclosed herein.

According to still another embodiment, a method for identifying a candidate mimetic of an isolated BBP-1, BBP-2, BBP-3, BBP-4, BBP-5, BBP-6, RhopH3, and/or ABRA polypeptide is provided. The method involves: providing a Band 3 molecule (e.g., the above-described isolated Band 3 peptides) which binds an isolated BBP-1, BBP-2, BBP-3, BBP-4, BBP-5, BBP-6, RhopH3, and/or ABRA polypeptide (e.g., a BBP-1, BBP-2, BBP-3, BBP-4, BBP-5, BBP-6, RhopH3, and/or ABRA polypeptide having SEQ ID NOs:46-53); contacting the Band 3 molecule with the BBP-1, BBP-2, BBP-3, BBP-4, BBP-5, BBP-6, RhopH3, and/or ABRA polypeptide in the presence or absence of a test molecule; and determining the binding of the test molecule to the Band 3 molecule, wherein a test molecule which binds to the Band 3 molecule and inhibits binding of the Band 3 molecule with the isolated peptide is a candidate mimetic of the isolated peptide.

According to still another aspect of the invention, an antibody (or fragment thereof) (referred to herein as a “anti-BBP-1, anti-BBP-2, anti-BBP-3, anti-BBP-4, anti-BBP-5, anti-BBP-6, anti-RhopH3, and/or anti-ABRA antibody”) that: (1) selectively binds to any of the above-described peptides, particularly the peptides having an amino acid sequence selected from the group consisting of SEQ ID Nos. 46-53; and (2) inhibits binding of the BBP-1, BBP-2, BBP-3, BBP-4, BBP-5, BBP-6, RhopH3, and/or ABRA polypeptide to Band 3 protein (particularly the Band 3 peptides disclosed herein) is provided. Preferably, the antibody is a monoclonal antibody, and, more preferably, a humanized monoclonal antibody.

The present invention also, in some aspects, involves the identification of cDNAs that encode Plasmodium polypeptides BBP-1, BBP-2, BBP-3, BBP-4, BBP-5, and BBP-6. The sequence of the coding portion of the Plasmodium gene for each is presented as SEQ ID NOs:38-43, and the predicted amino acid sequences of these genes' protein products are presented as SEQ ID NOs:46-51.

The invention thus involves in one aspect Plasmodium BBP-1, BBP-2, BBP-3, BBP-4, BBP-5, and BBP-6 polypeptides, nucleic acid molecules encoding those proteins, functional modifications and variants of the foregoing, usefil fragments of the foregoing, as well as therapeutic and diagnostic products (including antibodies), non-human animal models, and methods relating thereto.

According to one aspect of the invention, isolated nucleic acid molecules are provided. The isolated nucleic acid molecule is selected from the group consisting of: An isolated nucleic acid molecule selected from the group consisting of:

(a) nucleic acid molecules which hybridize under stringent conditions to a nucleic acid molecule comprising a nucleotide sequence selected from the group consisting of SEQ ID NOs:38-43 and which codes for aPlasmodium polypeptide,

(b) deletions, additions and substitutions of the nucleic acid molecules of (a), which code for a Plasmodium polypeptide,

(c) nucleic acid molecules that differ from the nucleic acid molecules of (a) or (b) in codon sequence due to the degeneracy of the genetic code, and

(d) complements of (a), (b) or (c).

The preferred isolated nucleic acids of the invention are Plasmodium nucleic acid molecules which encode a Plasmodium polypeptide. As used herein, a Plasmodium polypeptide refers to a protein that is encoded by a nucleic acid having SEQ ID NOs:38-45 and 54-59 or a functional fragment thereof, or a functional equivalent thereof (e.g., a nucleic acid sequence encoding the same protein as encoded by SEQ ID NOs:38-45 and 54-59), provided that the functional fragment or equivalent encodes a protein which exhibits a Plasmodium polypeptide functional activity. As used herein, a Plasmodium functional activity refers to the ability of a Plasmodium polypeptide of the invention to interact with a Band 3 molecule of the invention.

In the preferred embodiments, the isolated nucleic acid molecule is selected from the group consisting of SEQ ID NOs:38-45 and 54-59.

The invention provides isolated nucleic acid molecules which code for Plasmodium proteins and which hybridize under stringent conditions to a nucleic acid molecule consisting of the nucleotide selected from the group consisting of SEQ ID NOs:38-45 and 54-59. Such nucleic acids may be DNA, RNA, composed of mixed deoxyribonucleotides and ribonucleotides, or may incorporate synthetic non-natural nucleotides. Various methods for determining the expression of a nucleic acid and/or a polypeptide in cells are known to those of skill in the art and are described further below. As used herein, the term protein is meant to include large molecular weight proteins and peptides and low molecular weight peptides or fragments thereof.

The term “stringent conditions” as used herein refers to parameters with which the art is familiar. Nucleic acid hybridization parameters may be found in references which compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. More specifically, stringent conditions, as used herein, refers, for example, to hybridization at 65° C. in hybridization buffer (3.5× SSC, 0.02% Ficoll, 0.02% polyvinyl pyrrolidone, 0.02% Bovine Serum Albumin (BSA), 2.5 mM NaH₂PO₄ (pH 7), 0.5% SDS, 2 mM EDTA). SSC is 0.15M sodium chloride/0.15M sodium citrate, pH 7; SDS is sodium dodecyl sulphate; and EDTA is ethylenediaminetetracetic acid. After hybridization, the membrane upon which the DNA is transferred is washed at 2× SSC at room temperature, and then at 0.1× SSC/0.1× SDS at temperatures up to 68° C.

The foregoing set of hybridization conditions is but one example of stringent hybridization conditions known to one of ordinary skill in the art. There are other conditions, reagents, and so forth which can be used, which result in a stringent hybridization. The skilled artisan will be familiar with such conditions, and thus they are not given here. It will be understood, however, that the skilled artisan will be able to manipulate the conditions in a manner to permit the clear identification of homologs and alleles of Plasmodium nucleic acid molecules and Band 3 nucleic acid molecules of the invention. The skilled artisan also is familiar with the methodology for screening cells and libraries for expression of such molecules which then are routinely isolated, followed by isolation of the pertinent nucleic acid molecule and sequencing.

In general homologs and alleles of the malaria polypeptides typically will share at least 40% nucleotide identity and/or at least 50% amino acid identity to SEQ ID NOs:38-45 and 54-59 and SEQ ID NOs:46-53, 11-13, and 33-35, respectively, in some instances will share at least 50% nucleotide identity and/or at least 65% amino acid identity and in still other instances will share at least 60% nucleotide identity and/or at least 75% amino acid identity. Preferred homologs and alleles share nucleotide and amino acid identities with SEQ ID NOs:38-45, 54-59 and SEQ ID NOs:46-53, 11-13, and 33-35, respectively, and encode polypeptides of greater than 80%, more preferably greater than 90%, still more preferably greater than 95% and most preferably greater than 99% identity. The percent identity can be calculated using various publicly available software tools developed by NCBI (Bethesda, Md.) that can be obtained through the internet (ftp:/ncbi.nlm.nih.gov/pub/). Exemplary tools include the BLAST system available at

http://www.ncbi.nlm.nih.gov, which uses algorithms developed by Altschul et al. (Nucleic Acids Res. 25:3389-3402, 1997). Pairwise and ClustalW alignments (BLOSUM30 matrix setting) as well as Kyte-Doolittle hydropathic analysis can be obtained using the MacVector sequence analysis software (Oxford Molecular Group). Watson-Crick complements of the foregoing nucleic acid molecules also are embraced by the invention.

In screening for Plasmodium genes, a Southern blot may be performed using the foregoing conditions, together with a detectably labeled probe (e.g. radioactive or chemiluminescent probes). After washing the membrane to which the DNA is finally transferred, the membrane can be placed against X-ray film or a phosphorimager to detect the radioactive or chemiluminescent signal. In screening for the expression of Plasmodium RNA, Northern blot hybridizations using the foregoing conditions can be performed on samples taken from cells or subjects suspected of having a Plasmidium infection. Amplification protocols such as PCR using primers that hybridize to the sequences presented also can be used for detection of the Plasmodium genes or expression thereof.

Identification of related sequences can be achieved using PCR and other amplification techniques suitable for cloning related nucleic acid sequences. Preferably, PCR primers are selected to amplify portions of a nucleic acid sequence believed to be conserved (e.g., a binding domain, etc.). Again, nucleic acids are preferably amplified from a Plasmodium library.

The invention also includes degenerate nucleic acid molecules which include alternative codons to those present in the native materials. For example, serine residues are encoded by the codons TCA, AGT, TCC, TCG, TCT and AGC. Each of the six codons is equivalent for the purposes of encoding a serine residue. Thus, it will be apparent to one of ordinary skill in the art that any of the serine-encoding nucleotide triplets may be employed to direct the protein synthesis apparatus, in vitro or in vivo, to incorporate a serine residue into an elongating Plasmodium polypeptide. Similarly, nucleotide sequence triplets that encode other amino acid residues include, but are not limited to: CCA, CCC, CCG and CCT (proline codons); CGA, CGC, CGG, CGT, AGA and AGG (arginine codons); ACA, ACC, ACG and ACT (threonine codons); AAC and AAT (asparagine codons); and ATA, ATC and ATT (isoleucine codons). Other amino acid residues may be encoded similarly by multiple nucleotide sequences. Thus, the invention embraces degenerate nucleic acids that differ from the biologically isolated nucleic acids in codon sequence due to the degeneracy of the genetic code.

According to another aspect of the invention, further isolated nucleic acid molecules that are based on the above-noted Plasmodium nucleic acid molecules are provided. In this aspect, the isolated nucleic acid molecules are selected from the group consisting of:

(a) a unique fragment of the nucleotide sequence selected from the group consisting of:

nucleotides 1-1287 of SEQ ID NO:38 between 12 and 1286 nucleotides in length,

nucleotides 1-3576 of SEQ ID NO:39 between 12 and 3557 nucleotides in length,

nucleotides 1-903 of SEQ ID NO:40 between 12 and 902 nucleotides in length,

nucleotides 1-1203 of SEQ ID NO:41 between 12 and 1202 nucleotides in length,

nucleotides 1-3996 of SEQ ID NO:42 between 12 and 3995 nucleotides in length, and

nucleotides 1-876 of SEQ ID NO:43 between 12 and 875 nucleotides in length, and

nucleotides 1-2712 of SEQ ID NO:44 between 12 and 2711 nucleotides in length, and

nucleotides 1-2232 of SEQ ID NO:45 between 12 and 2231 nucleotides in length,

and nucleotides 1-1134 of SEQ ID NO:54 between 12 and 1133 nucleotides in length,

and nucleotides 1-1080 of SEQ ID NO:55 between 12 and 1079 nucleotides in length,

and nucleotides 1-660 of SEQ ID NO:56 between 12 and 659 nucleotides in length,

and nucleotides 1-1080 of SEQ ID NO:57 between 12 and 1079 nucleotides in length,

and nucleotides 1-1131 of SEQ ID NO:58 between 12 and 1130 nucleotides in length,

and nucleotides 1-343 of SEQ ID NO:59 between 12 and 342 nucleotides in length, and

(b) complements of (a),

wherein the unique fragments exclude nucleic acids having nucleotide sequences that are contained within SEQ ID NOs:38-45, 54-59, and that are known as of the filing date of this application.

The invention also provides isolated unique fragments of SEQ ID NOs:38-44 or complements of SEQ ID NOs:38-45, 54-59. A unique fragment is one that is a ‘signature’ for the larger nucleic acid. It, for example, is long enough to assure that its precise sequence is not found in molecules outside of the Plasmodium nucleic acid molecules defined above. Those of ordinary skill in the art may apply no more than routine procedures to determine if a fragment is unique within the Plasmodium. Unique fragments, however, exclude fragments completely composed of the nucleotide sequences that are contained within SEQ ID NO:38-45, 54-59 and that are known as of the filing date of this application.

Unique fragments can be used as probes in Southern blot, Northern blot, and Gene Chip/microarray assays to identify such nucleic acid molecules, or can be used in amplification assays such as those employing PCR. As known to those skilled in the art, large probes such as 200 nucleotides or more are preferred for certain uses such as Southern blots, while smaller fragments will be preferred for uses such as in PCR and Gene Chip/microarray assays. Unique fragments also can be used to produce fusion proteins for generating antibodies or determining binding of the polypeptide fragments, or for generating immunoassay components. Likewise, unique fragments can be employed to produce nonfused fragments of the Plasmodium polypeptides that are useful, for example, in the preparation of antibodies in immunoassays. Unique fragments further can be used as antisense molecules to inhibit the expression of Plasmodium nucleic acids and polypeptides, particularly for therapeutic purposes as described in greater detail below.

As will be recognized by those skilled in the art, the size of the unique fragment will depend upon its conservancy in the genetic code. Thus, some regions of SEQ ID NOs:38-44, 54-59 and their complements will require longer segments to be unique while others will require only short segments, typically between 12 and 32 nucleotides ormore in length (e.g. 12, 13, 14, 15, 16, 17, 18, 19, 20, 21, 22, 23, 24, 25, 26, 27, 28, 29, 30, 31, and 32 or more), up to the entire length of the disclosed sequence. Many segments of the polynucleotide coding region or complements thereof that are 18 or more nucleotides in length will be unique. Those skilled in the art are well versed in methods for selecting such sequences, typically on the basis of the ability of the unique fragment to selectively distinguish the sequence of interest from non-Plasmodium nucleic acid molecules. A comparison of the sequence of the fragment to those on known data bases typically is all that is necessary, although in vitro confirmatory hybridization and sequencing analysis may be performed.

A unique fragment can be a functional fragment. A functional fragment of a nucleic acid molecule of the invention is a fragment which retains some functional property of the larger nucleic acid molecule, such as coding for a functional polypeptide, binding to proteins, regulating transcription of operably linked nucleic acid molecules, and the like. One of ordinary skill in the art can readily determine using the assays described herein and those well known in the art to determine whether a fragment is a functional fragment of a nucleic acid molecule using no more than routine experimentation.

As used herein, the term “antisense oligonucleotide” or “antisense” describes an oligonucleotide that is an oligoribonucleotide, oligodeoxyribonucleotide, modified oligoribonucleotide, or modified oligodeoxyribonucleotide which hybridizes under physiological conditions to DNA comprising a particular gene or to a transcript of that gene and, thereby, inhibits the transcription of that gene and/or the translation of that mRNA. The antisense molecules are designed so as to interfere with transcription or translation of a target gene upon hybridization with the target gene or transcript. Those skilled in the art will recognize that the exact length of the antisense oligonucleotide and its degree of complementarity with its target will depend upon the specific target selected, including the sequence of the target and the particular bases which comprise that sequence. It is preferred that the antisense oligonucleotide be constructed and arranged so as to bind selectively with the target under physiological conditions, i.e., to hybridize substantially more to the target sequence than to any other sequence in the target cell under physiological conditions. Based upon SEQ ID NOs:38-45, 54-59, or upon allelic or homologous genomic and/or cDNA sequences, one of skill in the art can easily choose and synthesize any of a number of appropriate antisense molecules for use in accordance with the present invention. In order to be sufficiently selective and potent for inhibition, such antisense oligonucleotides should comprise at least 10 and, more preferably, at least 15 consecutive bases which are complementary to the target, although in certain cases modified oligonucleotides as short as 7 bases in length have been used successfully as antisense oligonucleotides (Wagner et al., Nature Biotechnology 14: 840-844, 1996).

Most preferably, the antisense oligonucleotides comprise a complementary sequence of 20-30 bases. Although oligonucleotides may be chosen which are antisense to any region of the gene or its transcripts, in preferred embodiments the antisense oligonucleotides correspond to N-terminal or 5′ upstream sites such as translation initiation, transcription initiation, or promoter sites. In addition, 3′-untranslated regions may be targeted. Targeting to mRNA splicing sites also has been used in the art but may be less preferred because alternative mRNA splicing of the Plasmodium transcript occurs. In addition, the antisense is targeted, preferably, to sites in which mRNA secondary structure is not expected (see, e.g., Sainio et al., Cell Mol. Neurobiol. 14(5):439-457, 1994) and at which proteins are not expected to bind. The present invention also provides for antisense oligonucleotides which are complementary to genomic DNA and/or cDNA corresponding to SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:54, SEQ ID NO:55, SEQ ID NO:56, SEQ ID NO:57, SEQ ID NO:58, and/or SEQ ID NO:59. Antisense to allelic or homologous cDNAs and genomic DNAs are enabled without undue experimentation.

In summary, the various aspects of the invention include one or more of the following utilities:

1. Treatment and prevention of malaria disease in human and animals:

(a) Development of a peptide, peptidomimetic, and/or protein antimalarial drugs partially or entirely based on the human Band 3 protein residues 720-761 and/or 807-826.

(b) Development of an antimalarial gene therapy using DNA plasmids encoding Band 3 protein sequence derived partially or entirely from the human Band 3 protein residues 720-761 and/or 807-826.

(c) Development of a peptide, peptidomimetic, and/or protein antimalarial drug using three dimensional structure information of human Band 3 protein containing a partial or the entire amino acid sequence of residues 720-761 and/or 807-826.

(d) Development of a non-peptide, non-protein, and/or non-peptidomimetic antimalarial drug derived from three dimensional structure information of human Band 3 protein containing a partial or the entire amino acid sequence of residues 720-761 and 807-826.

(e) Development of an idiotype protein vaccine that produces anti-idiotypic antibodies mimicking the entire or partial structure of residues 720-761 and/or 807-826 of the human Band 3 protein, for use in malaria disease.

(f) Development of an idiotype DNA vaccine in which DNA encodes the idiotypic determinants to induce the production of such anti-idiotypic antibodies as defined above.

(g) Use of a non-human erythrocyte Band 3 gene and/or protein sequence corresponding to the human Band 3 protein residues 720-761 and/or 807-826 for the purpose of developing drug or vaccine for human and/or animal malaria disease.

(h) Use of a gene and/or protein sequence corresponding to the MSP-1, BBP-1, BBP-2, BBP-3, BBP-4, BBP-5, BBP-6, RhopH3, ABRA molecules of the invention for the purpose of developing drugs or vaccines for human and/or animal malaria disease and for malaria diagnostic purposes.

2. Screening of the malaria parasite ligand(s) binding to the host Band 3 receptor:

(a) Use of a peptide and/or protein containing partial or entire sequence of human Band 3 protein residues 720-761 and/or 807-826 in efforts to identify and/or develop drug or vaccine for human and/or animal malaria disease. These include experiments including, but not limited to: protein or peptide binding experiments carried out in vitro and in vivo; three-dimensional structure-based approaches; computer modeling; combinatorial chemistry screening; other high throughput screening approaches. The malaria parasite ligand(s) identified and/or characterized by utilizing the inventions disclosed herein as new targets for the development of a highly efficient malaria vaccine and/or drug.

(b) Use of nucleotide sequence encoding partial or entire amino acid sequence of human Band 3 protein residues 720-761 and/or 807-826 to identify and/or functionally characterize the malaria parasite ligand(s) binding to the erythrocyte Band 3 protein.

(c) Use of a non-human erythrocyte Band 3 gene and/or protein sequence corresponding to the human Band 3 protein residues 720-761 and/or 807-826 for the purpose of carrying out the screening of malaria parasite ligand as described in (a) and (b) above.

3. Screening Assays to select agents which inhibit MSP1 binding to Band 3 protein, and screening assays to select agents that inhibit BBP-1, BBP-2, BBP-3, BBP-4, BBP-5, BBP-6, RhopH3, and/or ABRA binding to Band 3 protein.

(a) Use of a peptide and/or protein containing partial or entire sequence of MSP1, BBP-1, BBP-2, BBP-3, BBP-4, BBP-5, BBP-6, RhopH3, and/or ABRA polypeptide in combination with a peptide and/or protein containing human Band 3 protein (e.g., Band 3 residues 720-761 and/or 807-826) to identify and/or develop drug or vaccine for human and/or animal malaria disease. These include experiments including, but not limited to: protein or peptide binding experiments carried out in vitro and/or in vivo; three-dimensional structure-based approaches; computer modeling; combinatorial chemistry screening; other high throughput screening approaches. These screening assays can be used to detect lead molecules in mixtures (e.g., libraries) of synthetic or naturally-occurring molecules.

### EXAMPLES

Introduction to the Examples

To extend our observation that the erythrocyte Band 3 (−/−)mice are completely resistant to invasion by murine malaria parasite Plasmodium yoelii 17 XL, we have conducted a series of experiments to identify the key host receptor that mediates malaria parasite invasion into human red blood cells. Using a peptide scanning technique, we have identified two specific regions of the erythroid Band 3 protein (also known as Anion Exchanger 1 or AE1) that serve as the receptor for malaria parasite invasion into the human erythrocytes. These two regions are located within ectoplasmic domains 5 and 6 of the Band 3 protein. In our study, the ectoplasmic domains 5 and 6 are defined as below based on the published human Band 3 amino acid sequence

Ectoplasmic domain 5 (amino acid residues 720-761): (SEQ ID NO:22) GMPWLSATTVRSVTHANALTVMGKASTPGAAAQIQEVKEQRI

Ectoplasmic domain 6 (amino acid residues 807-857): (SEQ ID NO:23) DRILLLFKPPKYHPDVPYVKRVKTWRMHLFTGIQIICLAVLWVVKSTPASL

Four peptides (B3_{5A}, B3_{5B}, B3_{5C}, B3_{6A}) derived from ectoplasmic domains 5 and 6 of the Band 3 protein inhibited the invasion of the most lethal Plasmodium falciparum malaria parasite into human erythrocytes in vitro, while other peptides used in the scanning experiment had no significant effect as compared to the control sample. The peptides B3_{5C} and B3_{6A}showed highest inhibition amongst all peptides tested. The amino acid sequences of the four peptides are:

B3_{5A} (720-739): GMPWLSATTVRSVTHANALT (SEQ ID NO:1)

B3_{5B} (731-750): SVTHANALTVMGKASTPGAA (SEQ ID NO:2)

B3_{5C} (742-761): GKASTPGAAAQIQEVKEQRI (SEQ ID NO:3)

B3_{6A} (807-826): DRILLLFKPPKYHPDVPYVK (SEQ ID NO:4)

The blot overlay technique using the peptides B3_{5C} and B3_{6A}revealed that these peptides specifically bind to a set of human P. falciparum malaria proteins expressed at the merozoite stage. One of the proteins identified by the blot overlay assay corresponds to the molecular mass of Merozoite Surface Protein-1 (MSP-1), a well known malaria parasite protein.

We have further studied the interaction between the Band 3 peptides and MSP1 and have identified sequences within MSP1 which selectively bind to the Band 3 receptor. These studies and sequences are described in detail below. We have also examined the interaction between the Band 3 polypeptides and BBP-1, BBP-2, BBP-3, BBP-4, BBP-5, BBP-6, RhopH3, and ABRA polypeptides. These studies are described in more detail below. The amino acid sequences of the BBP-1, BBP-2, BBP-3, BBP-4, BBP-5, BBP-6, RhopH3, and ABRA polypeptides are SEQ ID NOs:46-53. These polypeptides of the invention are encoded by the nucleic acid molecules comprising nucleotide sequences selected from the group consisting of SEQ ID NOs:38-45.

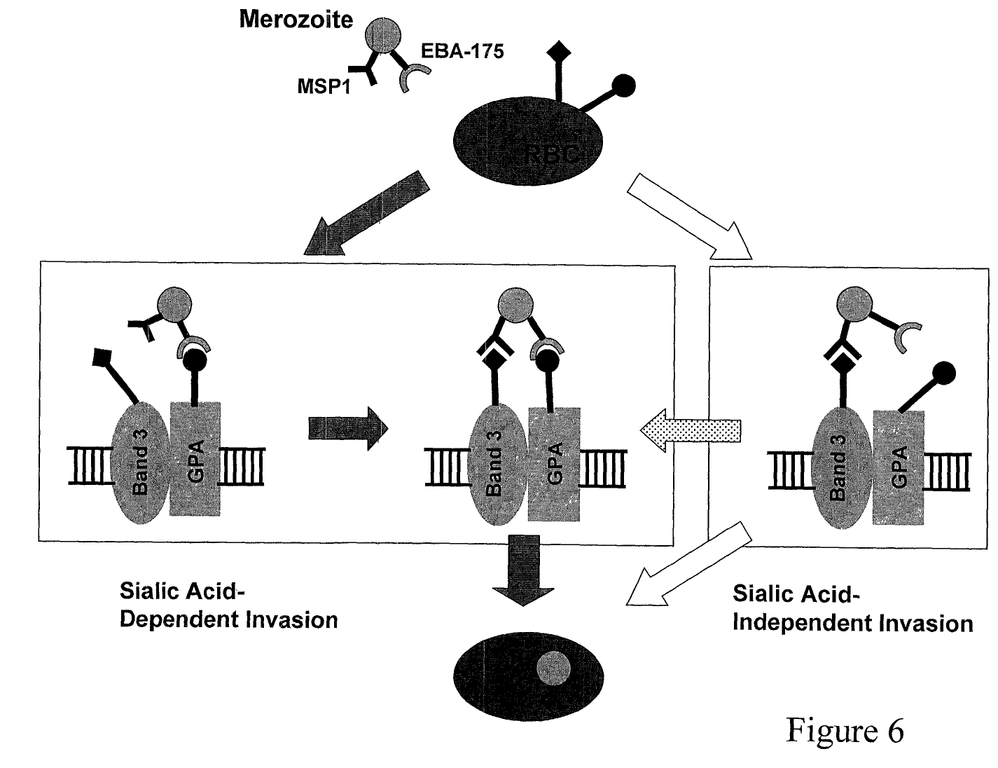

Based on our findings, we propose an parasite invasion pathway model illustrated in FIG. 6.

### Example 1

### Anti-Band 3 Antibody Preparation

Using the method of G. Kohler and C. Milstein, Nature 256, 495-496, (1975), a BALB/c mouse is immunized with washed human red cells expressing Band 3 or isolated Band 3 by weekly intraperitoneal administration of approximately 10⁷ erythrocytes each or an equivalent amount of the isolated Band 3 protein. The spleen of the mouse is then removed and a cell suspension prepared in tissue culture medium (RPMI-1640 with additional glutamine, 5 mM). The spleen cell suspension is mixed with a suspension of the mouse myeloma cell line P3/NSO-Ag4-1 (NS-0) (obtained from the ATCC) which, being deficient in the enzyme hypoxanthine-guanine phosphoribosyltransferase (HGPRT), will not grow in tissue culture media containing hypoxanthine, aminopterin and thymidine (HAT media).

The mixture contains four spleen cells to one myeloma cell. Fusion is promoted by the addition of polyethylene glycol (PEG) of an average molecular weight of 2000. After fusion, the cells are washed free of PEG, resuspended in HAT medium and allowed to grow to a density of 10⁶ live cells per ml and aliquots of 0.1 ml added to the wells of a 24-well tray containing feeder cells (from BALB/c thymus).

Partial changes of culture medium are performed at approximately 3, 5 and 7 days and the supernatants are removed approximately 14 days postfusion and tested for the presence of antibodies that bind to Band 3. Since the process is conducted in the presence of HAT, essentially non-fused myeloma cells are dead at this time, which prevents them from possibly overgrowing the fused (hybrid) ones.

The unfused spleen cells are also dead because of their very limited capacity to grow ex vivo in this tissue culture medium. The hybrid cells grow and multiply because the normal spleen cells contribute the enzyme HGPRT and the myelomatous cells for the capacity for indefinite proliferation. The supernatants from the wells containing colonies of hybrid cells are assayed on a panel including isolated Band 3 (e.g., immobilized in microtiter plate wells). The cellular contents of the positive well(s) that contain antibodies to Band 3 are then recovered and suspended to a concentration of 3 cells per ml.

Aliquots of 0.1 ml are then added to fresh wells, so that on the average only one of every three wells receives a cell and, thus, the colonies that result from the growth of this very diluted suspension are likely to be true “clones”, i.e., descendents from a single progenitor. When the colonies attained a size of 10² cells their supernatants are again screened for the presence of Band 3 antibodies and the most strongly positive ones are allowed to expand to a number of 10⁵ to 10⁶. Dilution of these cell suspensions to a concentration of 3 cells/ml and plating volumes of 0.1 ml as before results in the growth of doubly-cloned hybrid, antibody-producing cell lines.

The concentration of monoclonal antibody at the time of maturity of a culture flask is 20±/−0.5 μg/ml. The cloned hybridoma also grows in vivo in mice of the BALB/c strain or of its first generation (F1) hybrids. This growth is in the form of malignant myelomatous tumors. When live hybridoma cells are injected into susceptible animals, they secrete high concentrations of antibody into the peritoneal spaces. “Priming” the animals with irritants such as incomplete Freund's adjuvant or Pristane injected into the peritoneum, before grafting the hybridomas, results in the formation of large volumes of ascites containing antibody in concentrations higher than 5 mg/ml.

### Example 2

### Anti-Band 3 Antibody Blocks Penetration of P. falciparum Merozoites into Erythrocytes

The methods disclosed herein are based on those disclosed in U.S. Pat. No. 5,101,017, which reportedly are adapted from those described in L. H. Miller, S. J. Mason, J. A. Dvorak, T. Shiroishi and M. H. McGinnis, “Erythrocyte Receptors for Malarial Merozoites and the Duffy Blood Group System”, Human Blood Groups, 5th International Convocation on Immunology, Buffalo, N.Y., 1976, Basel Karger, pp. 394-400, 1977.

Standard numbers of merozoites are incubated with standard numbers of erythrocytes. In parallel wells, red cells of primates of different species are exposed to P. falciparum in the presence of an Anti-Band 3 antibody or non-specific antibody as a control, e.g., anti-Rh29 and anti-Kl4 antibodies (where are reactive with essentially all human and most primate red blood cells, but their antigenic epitopes are unrelated to Band 3). A third well for each erythrocyte donor contains only tissue culture medium instead of monoclonal antibodies. The two latter, control wells allow the determination of the proportion of red cells that are “normally” penetrated by the parasite under these conditions. Thus, comparison between this proportion and that in the well containing the anti-Band 3 monoclonal antibody permits estimation of its inhibitory effect

### Example 3

### Specificity Determination

The anti-Band 3 monoclonal antibody is tested for its capacity to immunoprecipitate or otherwise selectively bind to the Band 3 protein and/or Band 3 peptides of the invention (e.g., immobilized, labeled with a detectable agent). For example, red blood cell membrane proteins (including Band 3) or isolated Band 3 protein or peptides of the invention, separated by SDS-PAGE and blotted onto nitrocellulose filters, are exposed to labeled monoclonal antibody molecules and a single Band of the appropriate molecular size (of the Band 3 protein or peptides of the invention) and overall chemical characteristics is obtained.

### Example 4

### Preparation of Monoclonal Anti-idiotypic Antibodies

All antibody molecules are, at the same time, antigens since their ability to function as antibodies, i.e., to bind to antigen, depends on a special stereochemical configuration which is specific for each antibody and is called an “idiotype”. A monoclonal antibody immunoglobulin is constituted of exactly identical molecules, each having the same specific combining site, which, being complementary to the respective antigen, becomes antigenic for the antibody-producing host and to other animals of the same strain. In other words, the idiotype of an antibody leads to the production of anti-idiotypic antibodies. This antigenic property can thus be used to elicit such anti-idiotypic antibodies by injecting naive hosts with purified monoclonal antibodies produced in animals of the same inbred strain.

This procedure is based on that described in U.S. Pat. No. 5,101,017. The preparation of anti-idiotypic antibodies is accomplished by first purifying the original monoclonal antibody (designated Ab1) by affinity chromatography, emulsifying it in complete Freund's adjuvant and injecting this emulsion into the peritoneum and under the skin in multiple sites of BALb/c mice. A second, identical injection is given approximately two weeks later. Subsequent injections require the use of incomplete Freund's adjuvant. The schedule of these injections and the quantities of immunoglobulin injected are empirical and different procedures have been reported to be successful (e.g., two further injections in complete Freund's can be given two weeks apart and following the initial two injections in complete Freund's adjuvant by two weeks). The recipient mice are rested for two months followed by two bi-weekly injections of the Anti-Band 3 antibody (Ab1). One week later, the spleens are removed and fused with NSO myeloma cells. Hybridomas are grown as described elsewhere in this application and screening is performed by competitive inhibition of Ab1 binding to human red cells which express Band 3 or screening by competitive inhibition of Ab1 binding to isolated Band 3 or a Band 3 peptide of the invention. This inhibition test consists of adding the supernatants of hybridomas putatively producing anti-idiotypic antibodies (Ab2) to a dilution of Anti-Band 3 antibody (Ab1) and allowing the mixture to react with Band 3 (isolated or expressed by an erythrocyte or other cell). The presence of Ab2 inhibits that reaction. Confirmation of the specificity of presumptive anti-idiotypic antibody produced by the hybridomas is conducted by measuring its binding to red blood cells (there should be none) and the inhibition of monoclonal antibodies of the unrelated specificities: Rh29, K2, K14, M, N, B and Wr.sup.b (again there should be none). All these control tests being negative, the cells making the anti-anti-Band 3 antibody are cloned by limiting dilution.

These Ab2-producing clones (anti-anti-Band 3 ab) are then expanded and used to produce large amounts of supernatant and ascitic fluids. Ab2 binding to, and inhibition of the red cell penetration by, P. falciparum merozoites in subsequent experiments demonstrates that the epitope recognized by the anti-anti-Band 3 monoclonal antibody is indeed the site used by P. falciparum since the parasite shares the binding structure of the monoclonal antibody.

Thus, using standard immunological, combinatorial chemistry, and three dimensional structural approaches, novel compounds are identified that specifically inhibit the invasion of the malaria parasite into host erythrocytes. The development of these compounds is based on the structure of ectoplasmic domains 5 and 6 of the Band 3 protein, the receptor region as defined by the amino acid sequence of the peptides B3_{5A}, B3_{5B}, B3_{5C}, and B3_{6A}, and their corresponding MSP-1 ligand(s) on the merozoite surface.

### Example 5

### Band 3 is the Host Receptor Binding Merozoite Surface Protein-1 in the Malaria Parasite Plasmodium falciparum Invasion of Red Blood Cells

Methods

Synthetic Band 3 Peptides

Human Band 3 peptides were synthesized with an N-terminal biotin tag and purified to homogeneity by HPLC (Peptide 1, amino acid 424-435; 2, 477-491; 3A, 538-557; 3B, 551-570; 4A, 623-642; 4B, 634-653; 4C, 644-663; 5A, 720-739; 5B, 731-750; 5C, 742-761; 6A, 807-826; 6B, 823-842; 6C, 838-857). Peptides were initially solubilized with minimal DMSO and serially diluted with either PBS or pertinent buffer to give ≦1% DMSO final concentration in all subsequent assays. Peptide 6C was not soluble under these conditions and could not be used in the study.

Parasite Culture and Infection Determination

Plasmodium parasite cultures were maintained at 37° C. as described by Klotz, F. W., et. al., J Exp Med 165:1713-1718, 1987. To determine infection in RBCs, rings were counted from RBCs in Geimsa-stained thin smears.

Recombinant MSP1₃₈, MSP1₄₂ and MSP1₁₉

MSP1₃₈ (SEQ ID NO:33), MSP1₄₂ (SEQ ID NO:34), and MSP1₁₉ (SEQ ID NO:35) genes were amplified from a P. falciparum (FCB1 strain) cDNA library (J. B. Dame) by PCR using the following primers: 5′-CTCGAGCTCAGGATAAACCC (SEQ ID NO: 14, MSP1₃₈, sense, 3121-3133, Xhol), 5′-GCGGCCGCACTTGTTAGT (SEQ ID NO:15, MSP1₃₈, antisense, 4200-4193, NotI), 5′-CTCGAGCTGGAGAAGCAGTAACT (SEQ ID NO:16, MSP1₄₂, sense, 4201-4218, XhoI), 5′-GCGGCCGCACTAAATGAAACTGTATA (SEQ ID NO:17, MSP1₄₂, antisense, 5334-5321 NotI), 5′-CCGGGATCCAACATTTCACAACACCAA: (SEQ ID NO:18, MSP1₁₉, sense, 4993-5009 BamHI), 5′-CCGGAATTCAATGAAACTGTATAATA (SEQ ID NO:19, MSP1₁₉, antisense, 5334-5318, EcoRI). Similar cDNA libraries as well as CDNA pools are available to the public from MR4, ATCC (www.malaria.atcc.org).

Primer sequences were based on the Wellcome strain of P. falciparum MSP1 (Holder, A. A., et al., Nature 317:270-273, 1985; Miller, L. H., et al., Mol Biochem Parasitol 59:1-14, 1993). MSP1₃₈ and MSP1₄₂ were cloned into pGEX6P-2 (Amersham Pharmacia Biotech, Piscataway, N.J.), and MSP1₁₉ was cloned into pGEX-2TK (Amersham Pharmacia Biotech) using restriction sites indicated above. All three MSP1 domains were expressed as GST-fusion proteins in E. coli DH5α, affinity purified using GSH beads. ³²P-labeled MSP1₁₉ was obtained by radiolabeling GST-MSP1₁₉ with ³²P-γ-ATP and removing the GST domain with thrombin (as described in Manufacturer's instructions).

Recombinant Band 3 Peptides 5ABC, 5BC, and 6AB

The 5ABC (amino acid 720-761), 5BC (731-761), and 6AB (807-842) genes were amplified from a human reticulocyte cDNA library by PCR and cloned into pGEX-2TK (Pharmacia) as GST fusion proteins. Primers: 5′-CCGGGATCCGGGATGCCCTGGCTCAGTGCCA (SEQ ID NO:20, 5ABC, sense, 2272-2293, BamHI), 5′-CCGGAATTCTTAGATCCGCTGCTCTTTGACCTC (SEQ ID NO:21, 5ABC and 5BC, antisense, 2397-2377, EcoRI), 5′-CCGGGATCCTCCGTCACCCATGCCAACGCC (SEQ ID NO:44, 5BC, sense, 2305-2325, BamHI), 5′-CCGGGATCCGACCGCATCTTGCTTCTGTTCA (SEQ ID NO:25, 6AB, sense, 2533-2554, BamHI), and 5′-CCGGAATTCTTAGATCTGGATGCCCGTGAA (SEQ ID NO:26, 6AB, antisense, 2640-2620, EcoRI). GST-5ABC and GST-5BC were expressed, affinity purified, and radiolabeled with ³²P as above to obtain ³²P-labeled 5ABC and 5BC. GST-6AB expressed under same conditions was not soluble and could not be used in the study.

Blot Overlay Assay

Naturally released P. falciparum (3D7 strain) merozoites isolated as described (Mrema, J. E., et al., Exp Parasitol 54:285-295, 1982) and human RBC ghosts prepared as described (Dodge, J. T., et al., Arch Biochem Biophys 100:119-130, 1963) were subjected to SDS-PAGE. Proteins transferred onto nitrocellulose membrane were blocked overnight with 10% milk, 2% BSA, TBST (0.05%) at 4° C. After washing (4×) in TBST, the blot was incubated with biotinylated Band 3 peptides (each 400 μM) in TBST (0.025%), 10 mM phosphate buffer (pH 8.0), 60 mM KCl for 4 h at RT. After extensive washing, the blot was incubated with neutravidin-linked horseradish peroxidase (1:21,000, Pierce Chemical Co., Rockford, Ill.) in TBST (0.05%) with 2% BSA for 5-6 h at RT. After washing the blot with TBST (5×) and TBS (2×), bound peptides were visualized by the ECL method (Pierce Chemical Co.).

Native MSP1 Binding to mAb 5.2 and 5ABC

Radiolabeled parasite protein extract (180 μl) was incubated with either mAb 5.2 (20 μl) or GST-5ABC beads (40 μl, 50% slurry) for 22-24 h at 4° C. Protein G agarose beads (50 μl, 50% slurry) were added to the former mixture and further incubated for 3h. In both samples, beads were washed with PBS (2×) and proteins associated with beads were analyzed by SDS-PAGE followed by Coomassie staining and autoradiography. GST beads were used as control.

Yeast Two-Hybrid Assay

The 5ABC, 5BC, and 6AB genes were amplified by PCR as above and cloned into pGBKT7 (CLONTECH Laboratories, Inc., Palo Alto, Calif.). Primers were the same as the above except EcoRI (sense) and BamHI (antisense) restriction sites were used. MSP1₃₈ₐ, MSP1_{38b}, MSP1₄₂, and MSP1₁₉ constructs were prepared by amplifying the MSP1 gene by PCR using the same template as above and cloned into pGADT7 (Clontech). Primers used were: 5′-GGCCATATGGATGATACATCACATT (SEQ ID NO:27, MSP1₃₈ₐ, sense, 3148-3163, NdeI), 5′-GGCCTCGAGGTTTTCTAAACTGGCAT (SEQ ID NO:28, MSP1₃₈ₐ, antisense, 3780-3764, XhoI), 5′-GGCCATATGTTTAAAGTATTAAGTA (SEQ ID NO:29, MSP1_{38b}, sense, 3781-3796, NdeI), 5′-GGCCTCGAGTTCTCCTGTTACTACTTG (SEQ ID NO:30, MSP1_{38b}, antisense, 4206-4189, XhoI), 5′-GCCGAATTCGCAGTAACTCCTTCCG (SEQ ID NO:31, MSP1₄₂, sense, 4207-4222, EcoRI) 5′-GCCGGATCCAATGAAACTGTATAATA (SEQ ID NO:32, MSP1₄₂, antisense, 5334-5318, BamHI). PCR primers for MSP1₁₉ were the same as above except EcoRI (sense) and BamHI (antisense) restriction sites were used. Yeast two hybrid assays using these recombinant plasmids were carried out using MATCHMAKER yeast-two hybrid system 3 (CLONTECH Laboratories, Inc.), see Table 2 for summary of results.

Indirect Immunofluorescence Assay

RBCs freshly collected into citrate phosphate dextrose buffer were washed (3×) and resuspended in RPMI (20% hematocrit). Thin smears of RBCs on glass slides were allowed to air-dry and fixed in methanol (20-30 sec). The slides were washed with PBS (1×), blocked with 10% fetal bovine serum (FBS) in PBS (pH 7.4) for 1.5 h at 37° C., and again washed with PBS (5×, 10 min each). Fixed cells were incubated with GST-MSP1₄₂ (1 μM), GST-MSP1₃₈ (8 μM), or GST (9 μM) for 3.5 h at 37° C. in 10% FBS. Slide samples were washed with PBS (5×), incubated with goat anti-GST antibody (1:1,000, Amersham Pharmacia Biotech), washed again (5×), and incubated with rabbit anti-goat FITC-conjugated antibody (1:60, Sigma-Aldrich, St. Louis, Mo.). For visualizing spectrin, fixed cells treated with 10% FBS were incubated with a rabbit antibody raised against human spectrin (Sigma-Aldrich) followed by goat anti-rabbit FITC-conjugated antibody. All dilutions of proteins and antibodies were made in 10% FBS except FITC-conjugated antibodies.

Binding MSP1₃₈ and MSP1₁₉ to RBCs in Suspension

RBCs (500 μl of 50% suspension) were treated with Nm (3 mU, Clostridium perfringens, Roche) in 1 ml of RPMI (37° C., 1 h) or α-ChT (0.5 mg/ml, Sigma-Aldrich) in 1 ml RPMI (RT, 10 or 40 min) followed by PMSF (2 mM) for 30 min. To determine MSP1₃₈ binding, affinity purified GST-MSP1₃₈ was concentrated in PBS (pH 7.4) using Amicon spin column (10 kDa) and incubated with either Nm-treated or untreated human RBCs (7 μl packed volume) in PBS (pH 7.4, final 200 μl) at RT for 2 h. The mixture was passed through a bed of silicon oil (300 μl) by centrifugation. The RBC pellet was washed (1 ml) and resuspended (50-60 μl) in PBS and subjected to SDS-PAGE followed by Western blot using and anti-GST antibody (Amersham Pharmacia Biotech). GST was used as control. MSP1₁₉ binding: Enzyme-treated or untreated human and mouse RBCs (7 μl packed volume for human and 10 μl for mouse RBCs) were incubated with ³²P-labeled MSP1₁₉ in PBS as above. The incubation mixture was passed through silicon oil and RBCs were washed once as above. Radioactivity associated with resulting RBCs was measured using β-scintillation counter. Negative controls included samples with no RBCs and with only RBCs (no ³²P-labeled MSP1₁₉).

Solution Binding of MSP1₃₈ and MSP1₄₂

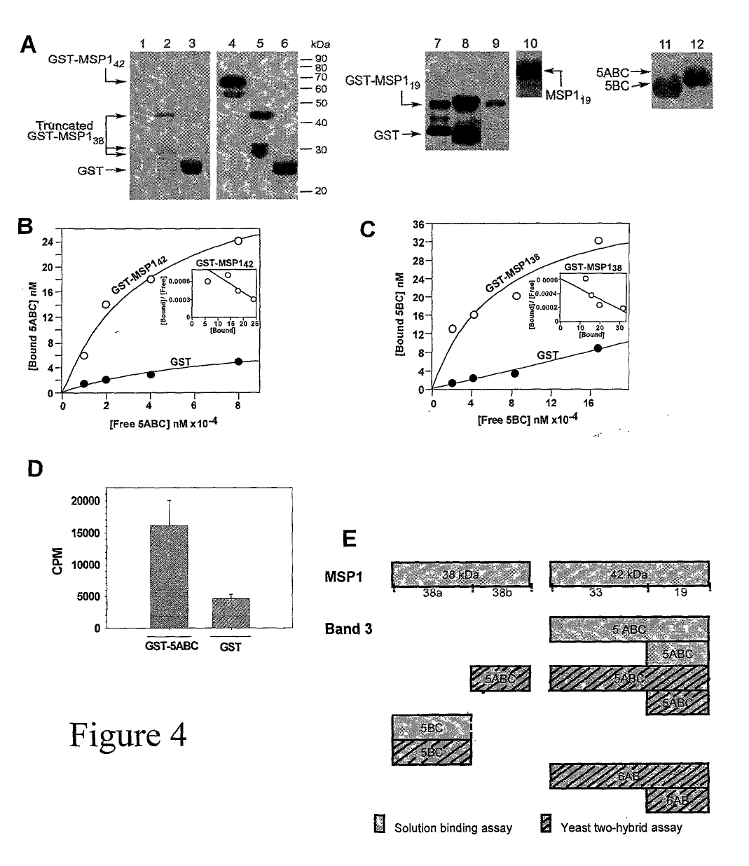

Binding assays were performed as described (Oh, S. S., et al., Mol Biochem Parasitol 108:237-247, 2000) using the following conditions: 20 mM phosphate buffer (pH 7.4), 120 mM NaCl, 1 mM DTT, 1.0 mg/ml BSA, 25° C., 3.5 h, 280 μl final vol. ³²P-labeled 5ABC (10, 20, 40, 80 μM) and 5BC (21, 42, 84, 168 μM) respectively bound to GST-MSP1₄₂ (FIG. 4B top panel) and GST-MSP1₃₈ (FIG. 4B middle panel) on beads in concentration-dependent manner. ³²P-labeled MSP1₁₉ bound specifically to the 5ABC domain (FIG. 4B bottom panel) as statistically analyzed by Student's t test. Binding to GST at comparable concentrations was not significant in all cases.

Metabolic Radiolabeling and Extraction of Parasite Proteins

Trophozoite-infected RBCs (60-100 μl packed volume) were purified on 70% Percoll gradient from a synchronized P. falciparum (3D7) culture and returned to culture in RPMI (without methionine, cysteine, and leucine) containing 15% human serum without adding fresh RBCs. ³⁵S-methionine and cysteine (3:1 mixture, 1.6 mCi, specific activity 1,175 Ci/mmol, NEN (PerkinElmer Life Sciences, Boston, Mass.) and ³H-leucine (250 μCi, specific activity 166 Ci/mmol, Amersham Pharmacia Biotech) was then added and the culture was kept for 12 hr. The radioactive medium was replaced with the cold RPMI with 15% serum, and incubation continued on shaker (60-80 rpm) until segmenters and released merozoites appeared in the culture (about 8 h). Pellets were collected initially from the culture (500 rpm, 7 min) and then from the resulting supernatant (3,500 rpm, 15 min). Combined pellets were stored at −80° C. or used immediately in the next step. Parasite pellets were treated with extraction buffer containing 50 mM Tris-HCl (pH 8.0), 5 mM EDTA, 5 mM EGTA, 150 mM NaCl, 0.5% Triton X-100, 0.5% BSA, 2 μg/ml aprotinin, 1 μg/ml of leupeptin, pepstatin A, bestatin, 10 mM PMSF and protease inhibitor cocktail (1×, Roche Molecular Biochemicals, Indianapolis, Ind.). The mixture was kept on ice for 1 hr and spun at 15,000 rpm for 15 min at 4° C. The supernatant was aliquoted and either used immediately or stored at −80° C. in binding assays.

Results

Band 3-Null RBCs Are Completely Resistant to P. falciparum Infection

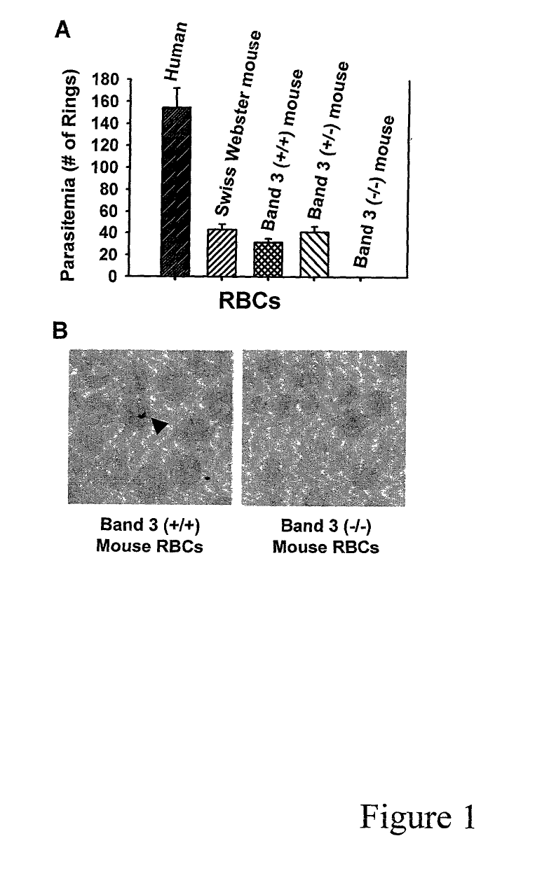

To investigate the role of Band 3 in the host RBC membrane during the P. falciparum invasion process, we tested the susceptibility of Band 3 null mouse RBCs to P. falciparum (3D7 strain) invasion in vitro. When infected with synchronized P. falciparum trophozoites, wild type and Band 3 (+/−) mouse RBCs showed a typical invasion profile after 24 hours (FIG. 1A) consistent with a previously study (Klotz, F. W., et al., J Exp Med 165:1713-1718, 1987). However, the Band 3 null RBCs did not show any new infection (rings) by P. falciparum. During the course of the experiment, Band 3 (−/−) mouse RBCs remained essentially intact in the culture as judged by the smears (FIG. 1B) suggesting that the increased fragility of these RBCs (Perkins, L. L., et al., Cell 86:917-927, 1996) could not have played a significant role in the outcome of this experiment.

We found protein 4.2 (−/−) mice (Peters, L. L., et al., J Clin Invest 103:1527-1537, 1999) developed parasitemia at a rate comparable to the wild type when challenged with P. yoelii 17XL (unpublished data). In view of these findings, the lack of Band 3 in the RBC membrane appears to be the primary cause for complete resistance to P. falciparum infection in our Band 3-null RBC model. We hypothesize that Band 3 is functioning as a host receptor independently or in conjunction with GPA during P. falciparum invasion into RBCs.

Band 3 Peptides Block P. falciparum Invasion into RBCs

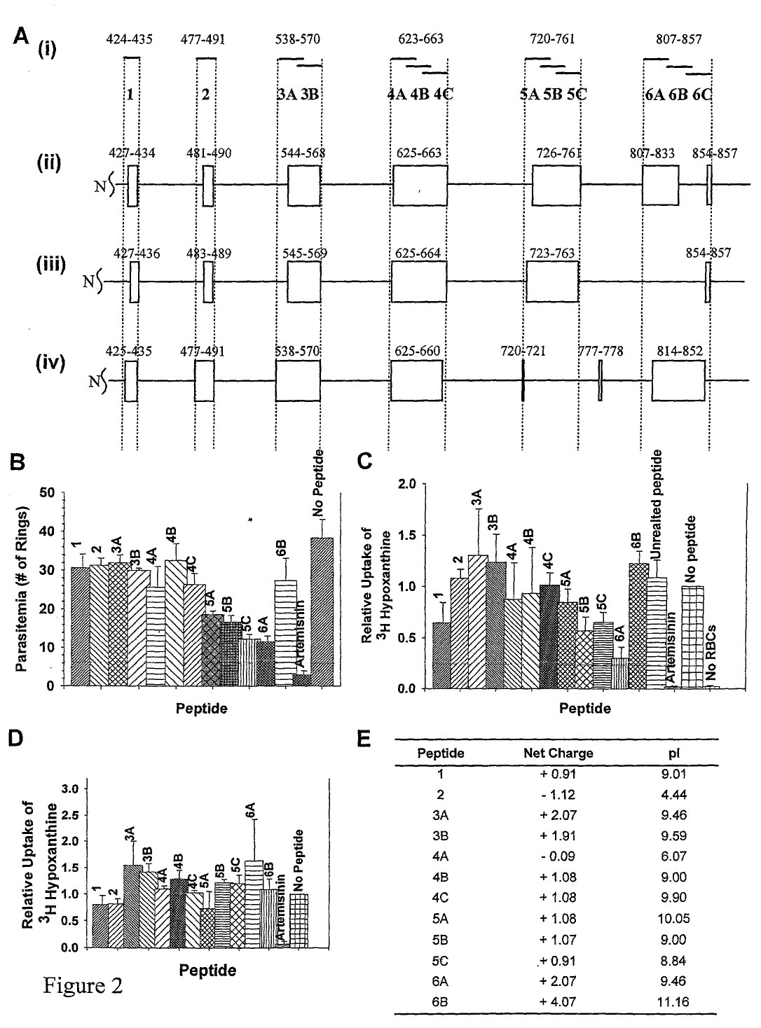

To investigate the possible role of Band 3 as a host receptor in P. falciparum invasion into RBCs, we employed a peptide scanning strategy based on recent topology models of human erythroid Band 3 (FIG. 2A). Synthetic peptides were derived from the putative ectodomains of human RBC Band 3, and their ability to inhibit the P. falciparum (3D7 strain) invasion of human RBCs in culture was tested using both visual counting (Schlichtherle, M., et al., Methods in Malaria Research 2000) and ³H-hypoxanthine incorporation (Chulay, J. D., et al., Exp Parasitol 55:138-146, 1983) methods. In the visual counting assay (FIG. 2B), Band 3 peptides 5A (p=0.016), 5B (p=0.013), 5C (p=0.006), and 6A (p=0.006) showed significant levels of inhibition of invasion at 500 μM concentration as compared to other Band 3 peptides and the control (no peptide). Inhibition by these four Band 3 peptides was concentration dependent (50, 200, 500, and 1000 μM peptides, data not shown). In the ³H-hypoxanthine uptake assay, peptide 6A (p=0.003) showed strong inhibition whereas peptides 1 (p=0.148), 5B (p=0.031) and 5C (p=0.023) showed moderate but significant levels of inhibition of invasion at 400 μM concentration as compared to other Band 3 peptides and control (no peptides and unrelated peptide) samples (FIG. 2C). Growth inhibition study using a similar ³H-hypoxanthine uptake method showed that peptides 5B (p=0.033), 5C (p=0.032) and 6A (p=0.478) added to the culture at 400 μM concentration did not affect the intraerythrocytic growth of the parasite (FIG. 2D). However, peptide 1 (p=0.320), 2 (p=0.140), and 5A (p=0.448) samples showed mild reduction in ₃H-hypoxanthine uptake as compared to control (no peptide) suggesting these peptides might be inhibiting parasite growth in culture. Thus, our study showed peptides 5B, 5C and 6A were the most effective inhibitors targeting P. falciparum invasion among all Band 3 peptides tested. Further, invasion blocking effects of peptides 5B, 5C, and 6A did not correlate with the net charge or pI of the peptides as these properties for peptides 4B, 1, and 3A, respectively were closely similar (FIG. 2E).

The IC₅₀ values (50% inhibition in the parasite invasion of RBCs) determined for peptide 5C and peptide 6A using the ³H-hypoxanthine incorporation method were 591±131 μM and 316±45 μM, respectively (mean of three experiments±standard error). The IC₅₀ values for peptides 5A and 5B could not be estimated because a saturable peptide concentration could not be reached with a low DMSO (≦1%) concentration. It is noteworthy that our peptides 5A, 5B, 5C, and 6A were designed by randomly dividing the two ectodomains of Band 3, and thus these peptides may not necessarily represent Band 3 amino acid compositions having the best inhibitory property. Our results, however, clearly demonstrate that peptides derived from two specific regions (amino acids 720-761 and 807-826) of human Band 3 inhibit the P. falciparum invasion of human RBCs at a significant level and in a concentration dependent manner. These findings are consistent with the view that Band 3 functions as an important receptor in the parasite invasion of RBCs.

Band 3 Peptides Interact with P. falciparum Merozoite Proteins

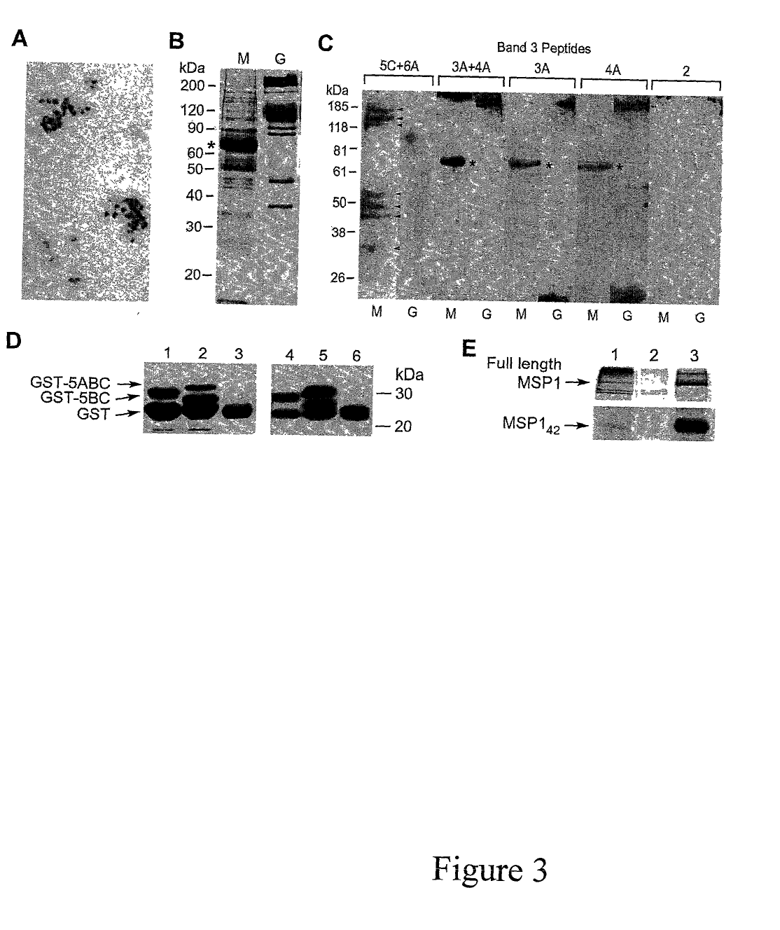

A binding study was carried out between the synthetic Band 3 peptides and merozoite proteins to understand the mechanism by which Band 3 peptides 5B, 5C, and 6A inhibit the parasite invasion of RBCs. P. falciparum merozoites essentially free of contaminating RBC membrane components were isolated (FIG. 3A), and total merozoite proteins were separated by SDS-PAGE (FIG. 3B). The purified merozoite protein mixture did not contain human RBC ghost proteins as judged by Coomassie blue staining. This was confirmed by Western blot using anti-spectrin and anti-Band 3 antibodies (not shown). A 1:1 mixture of peptides 5C and 6A showed specific binding to a number of merozoite proteins in the binding assay using a blot overlay method (FIG. 3C). Approximate molecular masses of these merozoite proteins are 175, 150, 125, 52, 48, 42, and 35 kDa (shown by arrowheads). In control samples, peptides 3A, 4A, and 2 did not show significant binding to any of these merozoite proteins, although a couple of weak signals were observed in the peptide 3A+4A sample. The peptide 5C+6A mixture did not show specific binding to RBC ghost proteins. Our blot overlay results provide evidence that Band 3 functions as a receptor in the P. falciparum invasion of RBCs, and suggest that the underlying mechanism for the observed inhibition of invasion involves a specific binding of the Band 3 peptides to one or more merozoite ligands, thus competitively blocking its interaction with the RBC Band 3 receptor.

Native MSP1 Binds to Recombinant Band 3

Among the merozoite proteins that specifically interacted with Band 3 peptides 5C and 6A the blot overlay binding assay (FIG. 3C) were polypeptides migrating at approximately 42 kDa and 35 kDa mass. To investigate whether at least one of the two polypeptides could be the 42 kDa or 38 kDa proteolytic fragment of MSP1 (MSP1₄₂ or MSP1₃₈), recombinant human Band 3 peptide 5ABC was prepared as a GST-fusion protein and affinity purified on GSH beads (FIG. 3D). P. falciparum proteins metabolically labeled with ³⁵S-methionine and ³H-leucine at the trophozoite stage were extracted from the mixture of late schizonts (segmenters) and naturally released merozoites using 0.5% Triton X-100. From this radiolabeled parasite protein extract, full-length MSP1 and MSP1₄₂ were immunoprecipitated with mAb 5.2 (MRA-94), a MSP1₁₉ (the 19 kDa C-terminal domain of MSP1)-specific monoclonal antibody raised against native P. falciparum MSP1 (FIG. 3E, lane 3). In parallel, full-length MSP1 and MSP1₄₂ in the total protein mixture also bound specifically to the 5ABC domain in the binding assay using GST-5ABC (lane 1) and GST (lane 2, control) beads. Similar to the blot overlay assay results, a few radioactive protein Bands were specifically associated with GST-5ABC beads (not shown).

Characterization of MSP1-Band 3 Interaction

During its maturation, P. falciparum MSP1 (full-length) is processed to give proteolytic fragments referred to as MSP1₈₃, MSP1₃₀, MSP1₃₈, and MSP1₄₂ which together form a non-covalent complex on the merozoite surface as merozoites are released into circulation from infected RBCs (Holder, A. A., et al., Mem Inst Oswaldo Cruz 3:37-42, 1992). MSP1₄₂ then undergoes secondary processing producing MSP1₁₉ (C-terminal domain of MSP1₄₂) that is retained on the merozoite surface and carried into the newly invaded RBC while all other MSP1 fragments are shed off by an unclear mechanism. To characterize the interaction of the Band 3 receptor with proteolytic fragments of MSP1, two independent binding studies were carried out in solution in vitro and in yeast two-hybrid system in vivo. For the in vitro binding study, P. falciparum MSP1₃₈, MSP1₄₂ and MSP1 ₁₉ (FIG. 4A) as well as human Band 3 peptides 5BC and 5ABC (FIG. 3D) were expressed in E. coli and purified as GST-fusion proteins. GST-MSP1₃₈ was purified as a mixture of three C-terminal truncated polypeptides. GST-MSP1₃₈, GST-MSP1₄₂ and GST-MSP1₁₉ reacted with P. falciparum MSP1 T9/94 rabbit antiserum (MRA-75) generated against full-length MSP1 in Western blot (not shown). Similarly, GST-MSP1₄₂ and GST-MSP1₁₉ reacted with mAb 5.2 (Western blot shown for GST-MSP1₁₉ in FIG. 4A, lane 9). Further, GST-MSP1₁₉, GST-5BC, and GST-5ABC were labeled with ³²P and treated with thrombin to afford pure ³²P-labeled MSP1₁₉ (FIG. 4A, lane 10), 5BC (lane 11), and 5ABC (lane 12).

In solution-binding assay, ³²P-labeled 5ABC and 5BC bound to GST-MSP1₄₂ (K_{d}=36 μM) and GST-MSP1₃₈ (K_{d}=67 μM), respectively, when neither showed significant binding to GST alone (FIGS. 4B, 4C, and 4D). Further, ³²P-labeled MSP1₁₉ bound to GST-5ABC at a significant level (p=0.027) as compared to the GST control sample. These results demonstrate specific binding interactions between the MSP1 domain and the Band 3 peptide domain. The three-dimensional structure for the 5ABC or 5BC region of Band 3 is not known. A difference of 11 amino acids in the primary structure of 5ABC and 5BC may be important for showing different binding property towards MSP1 in our in vitro experiments. What is clearly implicated from these results is that an ectoplasmic region of human Band 3 represented by amino acids 720-761 constitutes the binding site for MSP1₃₈, MSP1₄₂, and MSP1₁₉ presumably involving a number of specific binding interactions contained within this region.

In the second approach using a GAL4-based yeast two-hybrid assay, Band 3 peptides (5ABC, 5BC, 6AB) and MSP1 domains (MSP1₃₈ₐ, MSP1^{38b}, MSP1₄₂, MSP1₁₉) were expressed as a fusion to the GAL4 DNA-binding (DNA-BD) domain and GAL4 activation domain (AD), respectively. MSP1₃₈ was divided into MSP1₃₈ₐ and MSP1_{38b} in view of C-terminal truncated MSP138 used in the solution-binding assay. (See Table 2). Peptide 5ABC interacted with MSP1₄₂, MSP1₁₉, and MSP1_{38b}, peptide 5BC interacted with only MSP1₃₈ₐ, and peptide 6AB interacted with MSP1₄₂ and MSP1₁₉. For the yeast two-hybrid assay, the co-transformation method was used to analyze the protein interaction in yeast AH109 cells using SD/-Leu/-Trp selection plates. Activation of the MEL1 reporter gene upon specific binding of a MSP1 domain to a Band 3 peptide gave positive blue colonies using α-galactosidase assay. Plasmids expressing only the inserted gene of a Band 3 peptide or MSP1 domain did not undergo autonomous transcriptional activation of the reporter gene. No interaction was observed in other two-hybrid samples of Band 3 peptide and MSP1 domain. All positive and negative controls gave anticipated results. Specific interactions demonstrated with peptides 5ABC and 5BC in the yeast two-hybrid assay were consistent with the solution-binding assay results (FIGS. 4B, 4C, and 4D). The expression of 6AB as a soluble form in yeast cells (data not shown) provides support that the observed 6AB-MSP1₄₂ and 6AB-MSP1₁₉ interactions are specific. Binding results from two independent in vitro and in vivo methods summarized in FIG. 4E were remarkably similar indicating that a key function of MSP1₃₈, MSP1₄₂, and MSP1₁₉ is to interact with Band 3 in the RBC membrane during merozoite invasion of RBCs.

TABLE 2
Summary of Yeast Two-Hybrid Assay

| Fusion to DNA- | | |
|---|---|---|
| BD (pGBKT7 | Fusion to AD | Binding |
| vector) | (PgaDT7 vector) | Property |
| None | None | − |
| p53 | None | − |
| None | SV40 large T-antigen | − |
| p53 | SV40 large T-antigen | +++ |
| Lamin C | SV40 large T-antigen | − |
| 5ABC | SV40 large T-antigen | − |
| 5BC | SV40 large T-antigen | − |
| 6AB | SV40 large T-antigen | − |
| Lamin C | MSP1₃₈ₐ | − |
| Lamin C | MSP1_{38b} | − |
| Lamin C | MSP1₄₂ | − |
| p53 | MSP1₄₂ | − |
| p53 | MSP1₁₉ | − |
| 5ABC | MSP1₃₈ₐ | − |
| 5ABC | MSP1_{38b} | + |
| 5ABC | MSP1₄₂ | +++ |
| 5ABC | MSP1₁₉ | +++ |
| 5BC | MSP1₃₈ₐ | ++ |
| 5BC | MSP1_{38b} | − |
| 5BC | MSP1₄₂ | − |
| 5BC | MSP1₁₉ | − |
| 6AB | MSP1₃₈ₐ | − |
| 6AB | MSP1_{38b} | − |
| 6AB | MSP1₄₂ | +++ |
| 6AB | MSP1₁₉ | +++ |

Proteolytic Fragments of MSP1 Bind to RBCs in Sialic Acid-Independent Manner Earlier studies showed that native P. falciparum MSP1 (full-length) bound to RBCs in sialic acid-dependent manner (Perkins, M. E., et al., J Immunol 141:3190-3196, 1988; Su, S., et al., J Immunol 151:2309-2317, 1993). More recently, however, it has been shown that a number of peptides derived from MSP1₈₃, MSP1₃₈, and MSP1₄₂ bound to sialic acid-depleted RBCs with relatively high affinity (Urquiza, M., et al., Parasite Immunol 18:515-526, 1996). A recombinant segment (115 amino acids) of MSP1₃₈ referred to as p115MSP-1 also bound to wild-type human RBCs as well as En(a-) human RBCs lacking GPA (Nikodem, D., et al., Mol Biochem Parasitol 108:79-91, 2000).

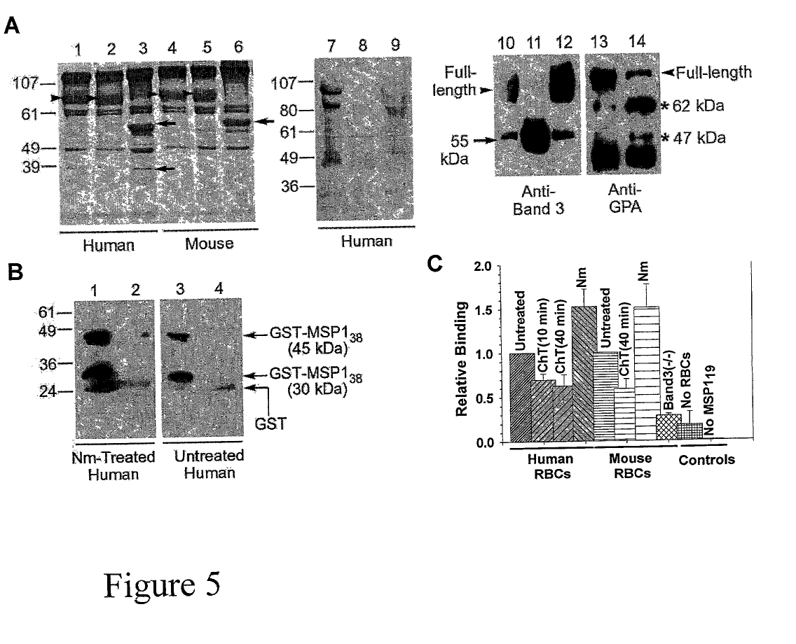

To further examine the RBC binding property of MSP1 proteolytic fragments, we first carried out a binding study using GST-MSP1₃₈ and intact human RBCs either untreated or pretreated with neuraminidase (Nm). Removal of sialic acids on the surface of Nm-treated RBCs was confirmed by periodic acid-Schiff (PAS) staining using RBC ghosts (FIG. 5A, lane 9). Both types of RBCs were incubated with purified GST-MSP1₃₈, sedimented through a bed of silicon oil as described (Nikodem, D., et al., Mol Biochem Parasitol 108:79-91, 2000) and subjected to SDS-PAGE followed by Western blotting using anti-GST antibody. Two truncated forms of GST-MSP1₃₈ (45 and 30 kDa Bands shown above in FIG. 4A, lane 5) bound to both treated (FIG. 5B, lanes 1) and untreated (lane 3) intact RBCs in suspension. GST alone did not bind to either type of RBCs (lanes 2, 4). These results demonstrate that MSP1₃₈ specifically interacted with the extracellular component of human RBCs in sialic acid-independent manner. This is consistent with our finding that peptide 5BC representing a non-glycosylated ectodomain of Band 3 bound to MSP1₃₈ in solution and MSP1₃₈ₐ in the yeast two-hybrid system.

In demonstrating the sialic acid-independent binding of MSP1₄₂ to intact RBCs, we used its C-terminal secondary processing fragment MSP1₁₉ known to be carried into newly invaded RBCs (Blackman, M. J., et al., J Exp Med 172:379-382, 1990). To carry out this study, we treated intact human and mouse RBCs with either chymotrypsin (ChT) or Nm (FIG. 5A). In ChT-treated human (FIG. 5A, lane 3) and mouse (lane 6) RBCs, full-length Band 3 (arrowheads) was digested into 55 kDa N-terminal and 38 kDa C-terminal fragments (arrows) as reported (Steck, T. L., et al., Biochemistry 17:1216-1222, 1978). The 38 kDa fragment known to be less stable than the 55 kDa fragment often appeared as a diffused faint Band in Coomassie gel (lane 6). As expected, Band 3 (arrowhead) was intact in Nm-treated human RBCs (lane 2) and mouse RBCs (lane 5). Western blotting of the human RBC ghost samples using anti-Band 3 antibody specific for the N-terminal cytoplasmic domain of human Band 3 confirmed these results (lanes 10-12). The extracellular region of GPA was also digested by ChT at an appreciable rate as evident in the PAS-stained gel (lane 8) and anti-GPA Western blot (lane 14) of human RBC samples. A significant level of sialic acid residues attached to ChT-digested GPA fragments was retained on the RBC surface as judged by PAS staining (land 8). The ChT-digested GPA fragments at approximately 62 kDa and 47 kDa molecular mass (shown with asterisks) in the human RBC sample (lane 14) were consistent with the previous report (Roggwiller, E., et al., Mol Biochem Parasitol 82:13-24, 1996).

These various types of RBCs were reacted with ³²P-labeled MSP1₁₉ (FIG. 4A, lane 10) in suspension, and the radioactivity associated with RBCs was analyzed. Human RBCs treated with ChT for 10 min and 40 min showed 30% (p=0.010) and 37% (p=0.040) reduction and with Nm (40 min) showed 52% increase (p=0.054) in their ability to bind MSP1₁₉ as compared to the untreated RBCs (FIG. 5C). In mouse RBC samples, ChT-treated RBCs (40 min) showed 41% reduction (p=0.005) and Nm-treated RBCs (40 min) showed 51% increase (p=0.086) as compared to the untreated RBCs. These results show for the first time that P. falciparum MSP1₁₉ binds to intact human and mouse RBCs in sialic acid-independent manner. Previously, a synthetic peptide referred to as peptide 5501 (20 amino acids) derived from the N-terminus of MSP1₁₉ was reported to bind human RBCs (Urquiza, M., et al., Parasite Immunol 18:515-526, 1996). Further, the reduced binding of MSP1₁₉ in ChT-treated RBC samples in our assays is directly correlated with alterations in Band 3 and GPA peptide backbones on the RBC surface. Nm treatment provided both human and mouse RBCs with increased ability to bind MSP1₁₉ demonstrating that the binding interaction does not involve sialic acids. Presumably, the increase in binding results from relatively unhindered access to the protein receptor upon removal of sialic acid residues from the RBC surface.

Band 3 is Important for P. falciparum MSP1 Binding to RBCs

Two approaches were considered to obtain evidence that Band 3 mediates sialic acid-independent interaction between MSP1 and RBCs. First, the above-described RBC binding assay was performed using ³²P-labeled MSP1₁₉ and intact Band 3 (−/−) mouse RBCs in suspension. As compared to untreated wild-type mouse RBCs, there was a 72% reduction (p=0.277×10⁻⁶) of radioactivity associated with Band 3 (−/−) mouse RBCs (FIG. 5C). However, since background radioactivity from the negative control sample containing no RBCs was about 18% of the positive control (untreated wild-type RBCs), the actual radioactivity associated with Band 3 (−/−) mouse RBCs was mere 10% above the background. Thus, intact Band 3 (−/−) mouse RBCs lacking both Band 3 and GPA from the plasma membrane showed a relatively insignificant level of binding to MSP1₁₉. In the second method, we carried out an indirect immunofluorescence assay (IFA) using wild-type human and mouse RBCs and Band 3 (−/−) mouse RBCs fixed in methanol. GST-MSP1₄₂ and GST-MSP1₃₈ (truncated forms) bound to human as well as mouse wild-type RBCs while GST alone did not, demonstrating that the observed binding was specific to the MSP1₄₂ and MSP1₃₈ domain, respectively. However, neither GST-MSP1₄₂, GST-MSP1₃₈, nor GST alone bound to Band 3 (−/−) mouse RBCs. In the indirect immunofluorescence assay of P. falciparum MSP1 binding to human and mouse RBCs, Anti-spectrin antibody staining confirmed all RBCs were morphologically normal.

Our truncated MSP1₃₈ (the 45 kDa GST-fusion protein) specifically bound to sialic acid-depleted RBCs (FIG. 5B), and the p 115MSP-1 construct substantially overlapping (at least about 60 amino acids) our MSP1₃₈ was shown to bind to intact human RBCs lacking the sialoglycoprotein GPA (Nikodem, D., et al., Mol Biochem Parasitol 108:79-91, 2000). In view of these findings, our IFA results demonstrate that the peptide backbone of Band 3 in the RBC membrane is important for binding MSP1₃₈ to the RBC surface. In this context, peptide 5ABC representing a putative ectoplasmic region (amino acids 720-761) of Band 3 specifically bound to MSP1₃₈ and MSP1₃₈a in our binding assays (FIG. 4E). This region of Band 3 shares 98% sequence identity between mouse and human. Our results thus indicate that P. falciparum MSP1₃₈ interacts with the Band 3 receptor on both human and mouse RBC surface during the parasite invasion of RBCs.

The MSP142 binding results in our IFA are consistent with the binding of its C-terminal fragment MSP1₁₉ to RBCs in suspension (FIG. 5C). Further, the level of MSP1₁₉ binding to RBCs decreased considerably with the limited ChT digestion of Band 3 and GPA peptide backbones on the RBC surface. Previously, ChT-treated human RBCs were shown to have marked reduction in invasion by P. falciparum as compared to untreated RBCs (Perkins, M., J Cell Biol 90:563-567, 1981). Since recombinant P. falciparum MSP1₄₂ and MSP1₁₉ bound to two distinct non-glycosylated ectoplasmic regions (5ABC and 6AB) of erythroid Band 3 (FIG. 4E) and native MSP1₄₂ specifically bound to 5ABC (FIG. 3E), our results taken together show that Band 3 functions as the receptor also for MSP1₄₂ and MSP1₁₉. A possibility that the peptide backbone of GPA might also play a secondary role in binding MSP1₄₂ and/or MSP1₁₉ to RBCs cannot be completely ruled out. Results from our RBC binding studies support the idea that Band 3 (−/−) RBCs are completely refractory to P. falciparum invasion due to the lack of an important interaction involving host Band 3 and proteolytic fragments of merozoite MSP1 such as MSP1₃₈, MSP1₄₂ and MSP1₁₉.

### Example 6

Identification of Plasmodium Polypeptides that Interact with Band 3 Methods

The “Bait” Construct

The bait construct containing the 5ABC domain (amino acids 720-761) of human Band 3 was prepared by a PCR amplification of the corresponding gene from a human reticulocyte cDNA library and cloning into a yeast two-hybrid vector pGBKT7 (Clontech) as a fusion to GAL4 DNA-binding (DNA-BD). Primers used were 5′-CCGGAATTCGGGATGCCCTGGCTCAGTGCCA-3′ (SEQ ID NO:36, sense, 2272-2293, EcoRI), 5′-CCGGGATCCTTAGATCCGCTGCTCTTTGACCTC-3′ (SEQ ID NO:37. antisense, 2397-2377, BamHI). The bait construct pGBKT7-5ABC was transformed into yeast AH109. The expression of 5ABC domain as a soluble fusion protein in yeast was confirmed by Western blotting of the cell lysate supernatant as described in the Clontech manual. The absence of autonomous transcriptional activation of the reporter gene by the bait domain 5ABC was confirmed on agar plates made with a minimal synthetic dropout medium (SD) lacking tryptophan (SD/-Trp), tryptophan and histidine (SD/-Trp-His), and tryptophan and adenine (SD/-Trp-Ade) using the X-α-Gal assay according to the Clontech manual.

Screening of the cDNA Library in Yeast Two-Hybrid System

The screening of P. falciparum (3D7 strain) cDNA library transformed into yeast PJ69-2A (Clontech) was performed by the standard yeast mating method using the bait construct pGBKT7-5ABC transformed into yeast Y187 (Clontech) as described in the Clontech manual (MATCHMAKER Two-Hybrid System 3). The mating mixture was spread onto 50 large (150 mm) plates of SD/-His-Leu-Trp (TDO). The cells were grown at 30° C. for 10 days. His⁺ colonies selected by this procedure were streaked onto SD/-His-Ade-Leu-Trp/X-a-Gal (QDO) plates and grown for 1 week at 30° C. Both positive (pGBKT7-53+pGADT7-T) and negative (pGBKT7+pGADT7) controls (Clontech) were included at each round of selection. Ade⁺, His⁺, Mel⁺ yeast colonies were selected from these plates for further analysis. Following overnight growth in the SD/-Leu-Trp-His broth at 30° C., cells from each clone were harvested. Plasmid DNAs isolated upon cell lysis were transformed into E. coli DH5α, and transformants were selected on LB/ampicillin plates. A selected colony from each transformation plate was subcultured overnight in the LB/ampicillin broth and the plasmid DNA was isolated by alkaline lysis. The nucleotide sequence of P. falciparum cDNA inserts was determined to identify these positive clones.

Results

Analysis of Positive Clones

From the cDNA library containing 9.8×10⁶ independent transformants, we selected 116 His⁺ colonies on SD/-His-Leu-Trp plates. These 116 His⁺ colonies were subjected to a second round of selection by streaking onto the SD/-Ade-His-Trp-Leu plate. Because the induction of the more tightly controlled GAL2-ADE2 reporter gene is required under these higher stringent selection conditions (Ade⁺), only 20 out of 116 colonies grew. Approximately 500 base pair sequence of P. falciparum cDNA inserts was determined from the 5′ end of the gene. Subsequent Blast analyses (at the NCBI and PlasmoDB website) of these insert cDNA sequences revealed that two of them were P. falciparum ABRA and RhopH3 (Table 3). All other cDNA insert sequences (total=18) were found only in PlasmoDB. Upon further analysis of the insert sequences, we found that only six of the eighteen insert cDNAs were non-redundant and in correct reading frame. These six were novel P. falciparum genes with no known functions associated with their gene products (Table 3). We have designated names to these six gene products as Band 3 Binding Protein (BBP)-1, 2, 3, 4, 5, and 6. The binding interaction between the Band 3 peptide 5ABC and each of the eight P. falciparum gene products was independently confirmed using the cotransformation and/or mating method (Clontech manual) in subsequent yeast two-hybrid assays under various stringency conditions as summarized in Table 3.

TABLE 3
Summary of cDNA library screening in yeast two-hybrid system

| BD-5ABC + | | -T- | -T-L- | -T-L- | -T-L-A- | Gene Name used by | Designated |
|---|---|---|---|---|---|---|---|
| AD-X^{a} | | L | H | A | H | PlasmoDB^{b} | Name^{c} |
| Clone | 8 | ++ | ++ | + | + | chr4_1.gen_205 | BBP-1 |
| Number | 12 | ++ | ++ | + | + | chr9_1.gen_311^{d} | RhopH3 |
| | 14 | ++ | ++ | + | + | chr12_1.gen_395^{e} | ABRA |
| | 48 | ++ | + | + | + | chrBLOB_004238.gen_2 | BBP-2 |
| | 59 | ++ | ++ | + | + | chr5_1.gen_122 | BBP-3 |
| | 74 | ++ | ++ | + | + | chr14_1.gen_490 | BBP-4 |
| | 94 | ++ | ++ | + | + | chr7_000072.gen_1 | BBP-5 |
| | 101 | ++ | ++ | + | + | cbr5_1.gen_79 | BBP-6 |

^{a}X denotes the clone number used in our cDNA library screening assay.
^{b}See attachment, Supplemental Material for Dataset 2: Description of genes by PlasmoDB.
^{c}BBP (Band 3 Binding Protein)-1, 2, 3, 4, 5, and 6 are names we have designated to respective gene products.
^{d}P. falciparum RhopH3, GenBank Accession No: M65059.
^{e}The 101 KDa P. falciparum malaria antigen (p101) termed Acidic Basic Repeat Antigen (ABRA), GenBank Accession No: J03902.

Table 4 depicts the Blast Sequence Homology results obtained for the Band 3 peptides having SEQ ID NOs: 1, 2, 3, and 4.

TABLE 4
BAND 3 BLAST HOMOLOGY SEOUENCES

SEQ ID NO. 1
P23562,NP_036783.1,CAA31128.1,NP_000333.1,CAA27555.1,NP_035533.1,AAA
37278.1,AAD43354.1,AAD43593.1,A30816 P15575,
P32847,AAF19584.2,AAG23156.1,AAG23157.1,AAG23155.1,AAF00977.1,NP_00
3031.1,AAF23240.1,XP_004678.1,NP_033233.1,AAG23158.1,AAC50964.1,NP_05

| | |
|---|---|
| 8744.1,AAF19583.2,AAB66833.1,AAC59881.1,AAG23154.1,P48746,O18917,NP_ | 4 |

033234.1,AAG25582.1,AAB05850.1,AAG25583.1,XP_002605.1,NP_058745.1,AA
D14330.1,NP_005061.1,S31828,CAA60670.1,Q9Z0S8,AAF50207.1,BAA34459.1,
NP_067505.1,NP_004849.1,NP_071341.1,BAB17922.1,AAF50207.1,AAF52496.1,
AAF52497.1,KADBID 004606,
AAA54840.1,AAA54837.1,AAA54839.1,NM_012651.1,X77738.1,NM_000342.1,
M27819.1,L35930.1,X12609.1,J02756.1,X03917.1,M29379.1,NM_011403.1,X0267
7.1,J04793.1,XM_008364.1,AC003043.1,AF163826.1,AF163828.1,AF163827.1,M1
9496.1,M23404.1,X61699.1,Z50848.1,U62531.1,U76669.2,AF012895.1,AF255774.
1,NM_009207.1,J04036.1,XM_004678.1,NM_003040.1,S45791.1,X62137.1,NM_0
17048.1,U48889.1,J05166.1,X03918.1,U20523.1,AF120099.1,M87060.1,AC009955
.4,NM_009208.1,M28383.1,AF031650.1,NM_017049.1,AF294651.1,J05167.1,S801
68.1,L27213.1,XM_002605.1,NM_005070.1,U05596.1,X87211.1,X70797.1,AF121
253.1,AE003550.2,AE003550.2,BF760317.1,BF724738.1,BF726058.1,BF726058.1,
BF726058.1,AW239627.1,BE255812.1,BE667859.1,BE683882.1,BE259443.1,BF7
60317.1,BF724738.1,BE683941.1,AA822979.1,BE512723.1,BF726058.1,AL12121
9.1,BE231685.1,AA362927.1,BF526005.1,F06947.1,AI592399.1,AI121401.1,BE68
3881.1,BF688963.1,BF688491.1,AW372960.1,AA755536.1,N58147.1,AW358179.
1,BE387636.1,AA979500.1,T86708.1,AC025326.3,AC010973.4,AC016330.5,AC01
6170.2,AC010044.5,AC014376.1,AL291529.1,I08446.1,E15207.1,I08447.1,AX001
285.1,AX001281.1,AX001279.1,NM_000342.1,XM_008364.1,NM_003040.1,XM_
004678.1,XM_002605.1,NM_005070.1,NP_000333.1,NP_003031.1,XP_004678.1,
XP_002605.1,NP_005061.1,NT_010755.1,
Seq ID NO:2
CAA31128.1, NP_000333.1, P23562, NP_036783.1, CAA27555.1, NP_035533.1,
AAA37278.1, AAD43354.1, AAD43593.1, AAF19584.2, AAG23156.1,
AAG23157.1, AAG23155.1, Q9Z058, AAF00977.1, NP_003031.1, AAF23240.1,
XP_004678.1, NP_033233.1, AAG23158.1, AAC50964.1, NP_058744.1,
AAF19583.2, AAB66833.1,, AAC59881.1, AAG23154.1, P48746, AAD14330.1,,
P15575, AAA48, AAB23405.1,, P32847, AAF50207.1, AAA54840.1,
AAA54837.1, AAA54839.1, X77738.1, NM_000342.1, AC003043.1, M27819.1,
L35930.1, X12609.1, XM_008364.1, J02756.1, NM_012651.1, X03917.1,
NM_011403.1, X02677.1, M29379.1, J04793.1, AF163826.1, AF163828.1,
AF163827.1, U20523.1, X03918.1, U62531.1, U76669.2, AF012895.1,
AF120099.1, AF121253.1, NM_009207.1, J04036.1, XM_004678.1, AF255774.1,
NM_003040.1, S45791.1, X62137.1, U488891, NM_017048.1, J05166.1,
S80168.1, M19496.1, M23404.1, BF760317.1, BF724738.1, BF724738.1,
BF724738.1, AL361787.1, BE512723.1, AA822979.1, BE255812.1, AL121219.1,
BE667859.1, BE259443.1, BF760317.1, BE868202.1, AI121401.1, BF724738.1,
BE231685.1, AW446011.1, AA362927.1, BF526005.1, I08446.1, E15207.1,
I08447.1, AC025326.3, AC010973.4, NP_000333.1, NM_0003421, XM_008364.1,
NT_010755.1,
SEQ ID NO:3
, CAA31128.1, NP_000333.1, P23562, NP_036783.1, CAA27555.1, NP_035533.1,
AAA37278.1, AAD43354.1, AAD43593.1, AAA54840.1, AAA54837.1,
AAA54839.1, X77738.1, NM_000342.1, M27819.1, AC003043.1, L35930.1,
X12609.1, XM_008364.1, NM_012651.1, J02756.1, M29379.1, NM_011403.1,
X03917.1, X02677.1, J04793.1, AF163826.1, AF163828.1, AF163827.1,
AC025326.3, I08446.1, E15207.1, I08447.1, NM_000342.1, XM_008364.1,
NP_000333.1, NT_010755.1,
SEQ ID NO:4
, 1BH7, CAA31128.1, NP_000333.1, P23562, NP_036783.1, CAA27555.1,
NP_035533.1, AAA37278.1, AAC59881.1,, P15575,, 211212, AAA48,
AAD43354.1, AAD43593.1, AAF19584.2, AAG23156.1, AAG23157.1,
AAG23155.1, AAF009771, NP_003031.1, AAF23240.1, XP_004678.1,
NP_033233.1, AAG23158.1, AAC50964.1, NP_058744.1, AAF19583.2,,
AAG23154.1, P48746, Q9Z0S8, NP_033234.1,, AAG25582.1, AAG25583.1,
NP_058745.1,, CAA99853.2,,, AAF52496.1, AAF52497.1,
AAF50207.1,KADBID 012974, AAA54840.1, AAA54837.1, AAA54839.1,
AAB15654.1, X77738.1, NM_000342.1, M27819.1, L35930.1, X12609.1,
AC003043.1, XM_0083641, NM_012651.1, J02756.1, X03917.1, M29379.1,
NM_011403.1, X02677.1, J04793.1, U48889.1, M19496.1, M23404.1, AF163826.1,
AF163828.1, AF163827.1, X03918.1, U62531.1, U76669.2, AF120099.1,
NM_009207.1, AF255774.1, J04036.1, XM_004678.1, NM_003040.1, S45791.1,
X62137.1, NM_017048.1, J05166.1, U20523.1, AF121253.1, AE003616.1,
BF748398.1, BF847884.1, BF910742.1, AI040499.1, N58147.1, AI071411.1,
AI178330.1, AW358923.1, W97645.1, AA067359.1, BF748398.1, AV607711.1,
BE870235.1, BE098805.1, H27406.1, BF688963.1, BE622988.1, AV602684.1,
AA622036.1, AA595190.1, AL121219.1, AI159996.1, BF847884.1, BE868202.1,
BF342604.1, AV604384.1, AW477680.1, BE663637.1, AV607712.1, AW446011.1,
AI870534.1, AI326582.1, R73348.1, BE873843.1, AI909314.1, AA359681.1,
BF910742.1, AW553811.1, C00728.1, AC025326.3, AC010973.4, AC015894.3,
AL328123.1, AZ616813.1, NC_001148.1, AE000426.1, I08446.1, I08447.1,
NP_000333.1, NM_0003421, XM_008364.1, NT_010755.1,

EQUIVALENTS

Those skilled in the art will recognize, or be able to ascertain using no more than routine experimentation, many equivalents to the specific embodiments of the invention described herein. Such equivalents are intended to be encompassed by the following claims.

All references disclosed herein are incorporated by reference in their entirety.

We claim:

1. An isolated Band 3 polypeptide comprising an amino acid sequence selected from the group consisting of SEQ ID NO. 1, 2, 3, and 4 as shown herein:
SEQ ID NO: 1: GMPWLSATTVRSVTHANALT (also referred to herein as sequence B3_{5A});
SEQ ID NO:2: SVTHANALTVMGKASTPGAA (also referred to herein as sequence B3_{5B});
SEQ ID NO:3: GKASTPGAAAQIQEVKEQRI (also referred to herein as sequence B3_{5C});
SEQ ID NO:4: DRILLLFKPPKYHPDVPYVK (also referred to herein as sequence B3_{6A}); and unique fragments thereof, wherein the unique fragments
(1) bind to an MSP-1 polypeptide and
(2) exclude the sequences set forth in Table 4:Band 3 Blast Homology Sequences.

2. An isolated nucleic acid molecule that encodes the isolated polypeptide of claim 1.

3. An expression vector comprising the isolated nucleic acid of claim 2 operably linked to a promoter.

4. A host cell transfected or transformed with an expression vector of claim 3.

5. An immunogenic composition comprising:
one or more isolated polypeptides of claim 1; and
a pharmaceutically acceptable carrier;
wherein the polypeptides are present in an effective amount to induce an immune system response.

6. The composition of claim 5, further comprising an adjuvant.

7. A method of making a medicament, comprising:
placing one or more isolated polypeptides of claim 1 in a pharmaceutically acceptable carrier.

8. A method for identifying a candidate mimetic of an isolated polypeptide of claim 1, comprising
providing an MSP-1 polypeptide which binds the isolated polypeptide of claim 1,
contacting the MSP-1 polypeptide with a test molecule, and
determining the binding of the test molecule to the MSP-1 polypeptide,
wherein a test molecule which binds to the MSP-1 polypeptide and inhibits binding of the MSP-1 polypeptide to the polypeptide of claim 1 is a candidate mimetic of the isolated polypeptide of claim 1.

9. A protein microarray comprising at least one isolated Band 3 polypeptide selected from the group consisting of SEQ ID NOS. 1, 2, 3, and 4.

10. An anti-Band 3 antibody or fragment thereof that selectively binds to a polypeptide of claim 1, wherein the antibody inhibits infection of cells by P. falciparum merozoite malaria parasite.

11. An anti-idiotype antibody which selectively binds to the idiotype of the antibody of claim 10.

12. A method for making an anti-idiotypic antibody comprising:
immunizing an animal with an antibody of claim 10 under conditions to elicit an immune system response to an idiotype of said antibody of claim 10.

13. A method for treating a malaria infection, comprising:
administering to a subject in need of such treatment, an effective amount of an anti-Band 3 antibody of claim 10 to treat the malaria infection.

14. A method for inducing an immune system response to treat a malaria infection, comprising:
administering to a subject in need of such treatment, an effective amount of an anti-Band 3 antibody of claim 10 under conditions to induce an anti-idiotypic immune response to the anti-Band 3 antibody idiotype.

15. A method for identifying a candidate mimetic of a MSP-1 polypeptide, comprising
providing an isolated Band 3 polypeptide which binds a MSP-1 polypeptide,
contacting the Band 3 polypeptide with a test molecule, and
determining the binding of the test molecule to the Band 3 polypeptide,
wherein a test molecule which binds to the isolated Band 3 polypeptide and inhibits binding of the Band 3 polypeptide to the MSP-1 polypeptide is a candidate mimetic of the MSP-1 polypeptide.

16. The method of claim 15, wherein the MSP-1 polypeptide has a sequence selected from the group consisting of SEQ ID NO:11, SEQ ID NO:12, SEQ ID NO:13, SEQ ID NO:33, SEQ ID NO. 34, and SEQ ID NO:35.

17. The method of claim 15, wherein the test molecule is an antibody.

18. An isolated polypeptide, wherein the polypeptide comprises an amino acid sequence selected from the group consisting of SEQ ID NOs:11, 12, 13, 33, 34, and 35, or fragments thereof.

19. A pharmaceutical composition comprising:
one or more isolated polypeptides of claim 18 and
a pharmaceutically acceptable carrier;
wherein the polypeptides are present in an effective amount to induce an immune system response.

20. The pharmaceutical composition of claim 19, further comprising an adjuvant.

21. A method of making a medicament, comprising:
placing one or more isolated polypeptides of claim 19 in a pharmaceutically acceptable carrier.

22. A method of preventing or treating a malaria infection, comprising administering a pharmaceutical composition of claim 19 to a subject in need of such treatment in an amount effective to prevent or treat the malaria infection.

23. A malaria polypeptide binding polypeptide that selectively binds to a isolated malaria polypeptide of claim 18, wherein the binding polypeptide is an antibody or antigen-binding fragment of an antibody.

24. A pharmaceutical composition comprising the malaria polypeptide binding polypeptide of claim 23, in a pharmaceutically acceptable carrier.

25. A method of preventing or treating a malaria infection, comprising administering the pharmaceutical composition of claim 24 to a subject in need of such treatment in an amount effective to prevent or treat the malaria infection.

26. An isolated nucleic acid, wherein the nucleic acid molecule comprises a nucleotide sequence selected from the group consisting of SEQ ID NOs:54-59, or fragments thereof.

27. An isolated Band 3 polypeptide, comprising an amino acid sequence selected from the group consisting of SEQ ID NO. 1, 2, 3, and 4 as shown herein:
SEQ ID NO: 1: GMPWLSATTVRSVTHANALT (also referred to herein as sequence B3_{5A});
SEQ ID NO:2: SVTHANALTVMGKASTPGAA (also referred to herein as sequence B3_{5B});
SEQ ID NO:3: GKASTPGAAAQIQEVKEQRI (also referred to herein as sequence B3_{5C});
SEQ ID NO:4: DRILLLFKPPKYHPDVPYVK (also referred to herein as sequence B3_{6A}), and unique fragments thereof, wherein the unique fragments
(1) bind to an isolated polypeptide comprising an amino acid sequence selected from the group consisting of SEQ ID NOs:46-53, or fragment thereof, and
(2) exclude the sequences set forth in Table 4:Band 3 Blast Homology Sequences.

28. An isolated nucleic acid molecule that encodes the isolated Band 3 polypeptide of claim 27.

29. An expression vector comprising the isolated nucleic acid of claim 29 operably linked to a promoter.

30. A host cell transfected or transformed with an expression vector of claim 29.

31. An immunogenic composition comprising:
one or more isolated Band 3 polypeptides of claim 27 and
a pharmaceutically acceptable carrier;
wherein the Band 3 polypeptides are present in an effective amount to induce an immune system response.

32. The composition of claim 31, further comprising an adjuvant.

33. A method of making a medicament, comprising:
placing one or more isolated Band 3 polypeptides of claim 27 in a pharmaceutically acceptable carrier.

34. A method for identifying a candidate mimetic of an isolated Band 3 polypeptide of claim 27, comprising
providing a malaria polypeptide comprising an amino acid sequence selected from the group consisting of SEQ ID NOs:46-53, or fragment thereof that binds the isolated Band 3 polypeptide or fragment thereof of claim 27,
contacting the malaria polypeptide or fragment thereof, with a test molecule, and
determining the binding of the test molecule to the malaria polypeptide or fragment thereof, wherein a test molecule which binds to the polypeptide or fragment thereof and inhibits binding of the isolated Band 3 polypeptide to the malaria polypeptide, is a candidate mimetic of the isolated Band 3 polypeptide of claim 27.

35. A method for identifying a candidate mimetic of an isolated malaria polypeptide, comprising
providing a Band 3 molecule which binds a malaria polypeptide comprising an amino acid sequence selected from the group consisting of SEQ ID NOs:46-53,
contacting the Band 3 molecule with a test molecule, and
determining the binding of the test molecule to the Band 3 molecule, wherein a test molecule which binds to the Band 3 molecule and inhibits binding of the malaria polypeptide with the Band 3 polypeptide is a candidate mimetic of the malaria polypeptide.

36. The method of claim 35, wherein the test molecule is an antibody.

37. An isolated polypeptide molecule comprising an amino acid sequence selected from the group consisting SEQ ID NOs:46-52.

38. A pharmaceutical composition comprising:
one or more isolated polypeptides of claim 37 and
a pharmaceutically acceptable carrier;
wherein the polypeptides are present in an effective amount to induce an immune system response.

39. The pharmaceutical composition of claim 38, further comprising an adjuvant.

40. A method of making a medicament, comprising:
placing one or more isolated polypeptides of claim 38 in a pharmaceutically acceptable carrier.

41. A method of preventing or treating a malaria infection, comprising administering a pharmaceutical composition of claim 38 to a subject in need of such treatment in an amount effective to prevent or treat the malaria infection.

42. A malaria polypeptide binding polypeptide that selectively binds to a isolated malaria polypeptide of claim 37, wherein the binding polypeptide is an antibody or antigen-binding fragment of an antibody.

43. A pharmaceutical composition comprising the malaria polypeptide binding polypeptide of claim 42, in a pharmaceutically acceptable carrier.

44. A method of preventing or treating a malaria infection, comprising administering the pharmaceutical composition of claim 43 to a subject in need of such treatment in an amount effective to prevent or treat the malaria infection.

45. An isolated nucleic acid molecule selected from the group consisting of:
(a) nucleic acid molecules which hybridize under stringent conditions to a nucleic acid molecule comprising a nucleotide sequence selected from the group consisting of SEQ ID NO:38-44 and which codes for a Plasmodium polypeptide,
(b) deletions, additions and substitutions of the nucleic acid molecules of (a), which code for a Plasmodium polypeptide,
(c) nucleic acid molecules that differ from the nucleic acid molecules of (a) or (b) in codon sequence due to the degeneracy of the genetic code, and
(d) complements of (a), (b) or (c).

46. The isolated nucleic acid molecule of claim 45, wherein the isolated nucleic acid molecule comprises a nucleotide sequence selected from the group consisting of SEQ ID NO:38-44.

47. An isolated nucleic acid molecule selected from the group consisting of:
(a) a unique fragment of the nucleotide sequence selected from the group consisting of:
nucleotides 1-1287 of SEQ ID NO:38 between 12 and 1286 nucleotides in length,
nucleotides 1-3576 of SEQ ID NO:39 between 12 and 3557 nucleotides in length,
nucleotides 1-903 of SEQ ID NO:40 between 12 and 902 nucleotides in length,
nucleotides 1-1203 of SEQ ID NO:41 between 12 and 1202 nucleotides in length,
nucleotides 1-3996 of SEQ ID NO:42 between 12 and 3995 nucleotides in length, and
nucleotides 1-876 of SEQ ID NO:43 between 12 and 875 nucleotides in length, and
nucleotides 1-2712 of SEQ ID NO:44 between 12 and 2711 nucleotides in length, and
(b) complements of (a),
wherein the unique fragments exclude nucleic acids having nucleotide sequences that are contained within SEQ ID NO:38-44, and that are known as of the filing date of this application.

48. An expression vector comprising the isolated nucleic acid molecule of claim 46 operably linked to a promoter.

49. An isolated polypeptide molecule comprising a unique fragment of amino acid sequence SEQ ID NO:53 that binds to a Band 3 polypeptide.
